(19) United States
(12) Patent Application Publication
(10) Pub. No.: US 2003/0181371 A1
(43) Pub. Date: Sep. 25, 2003

(54) Compositions and methods of using collajolie

(75) Inventors: William L. Hunter, Vancouver (CA); David M. Gravett, Vancouver (CA); Philip M. Toleikis, Vancouver (CA); Arpita Maiti, Vancouver (CA)

Correspondence Address: SEED INTELLECTUAL PROPERTY LAW GROUP PLLC, 701 FIFTH AVE, SUITE 6300, SEATTLE, WA, 98104-7092, US

(73) Assignee: Angiotech Pharmaceuticals, Inc., Vancouver (CA)

(21) Appl. No.: 10/331,125

(22) Filed: Dec. 27, 2002

Related U.S. Application Data
(60) Provisional application No. 60/344,568, filed on Dec. 28, 2001.

(51) Int. Cl. A61K 38/39; A61K 31/65; A61K 31/19

(52) U.S. Cl. 514/12; 514/154; 514/575

(57) ABSTRACT

Compositions comprising collagen and at least one metalloprotease inhibitor, and methods of making and using same are provided.

### CROSS-REFERENCE TO RELATED APPLICATION

This application claims the benefit of U.S. Provisional Patent Application No. 60/344,568, filed Dec. 28, 2001, where this provisional application is incorporated herein by reference in its entirety.

### BACKGROUND OF THE INVENTION

1. Field of the Invention

The present invention relates generally to pharmaceutical compositions and methods, and more specifically, to compositions and methods for enhancing the duration of activity of implanted collagen materials.

2. Description of the Related Art

Collagen is one of the most abundant proteins in mammals, representing up to 30% of the dry weight of the human body (see, L. C. Junqueira and J. Carneiro, Basic Histology, 4th ed., Lange Medical Publications, Los Altos, Calif., 1983, pp. 89-119). Collagen provides strength and flexibility for skin, hair and nails, and is also a major and essential component of muscles, tendons, cartilage, ligaments, joints and blood vessels.

Collagen can be found in at least five different naturally occurring forms that are produced by several different cell types. Type I collagen is the most abundant form of collagen, and can be found throughout the body. It is produced by fibroblasts, osteoblasts, odontoblasts, and chondroblasts, and can be found in bones, dentin, dermis, and fibrous cartilage. Type II collagen is produced by chondroblasts, and can be found primarily in cartilage. Type III collagen is produced by smooth muscle fibroblasts, reticular cells, Schwann cells, and hepatocytes. Its primary function is to maintain the structure of organs, and can be found in smooth muscles, endoneurium, arteries, uterus, liver, spleen, kidney, and lung tissue.

Type IV collagen is primarily believed to be involved in support and filtration, and can be found in the epithelial and endothelial basal lamina and basement membranes. Type V collagen is found in fetal membranes, blood vessels, and placental basement membrane,

Collagen has been suggested for use in the treatment of a variety of medical applications, including for example, cosmetic surgery, arthritis, skin regeneration, implants, organ replacement, and treatment for wounds and bums (see e.g, U.S. Pat. Nos. 6,309,670, 5,925,736, 5,856,446, 5,843,445, 5,800,811, 5,783,188, 5,720,955, 5,383,930, 5,106,949, 5,104,660, 5,081,106, 4,837,379, 4,604,346, 4,485,097, 4,546,500, 4,539,716, and 4,409,332).

Collagen however, has presented several problems associated with medical applications. For example, in the context of implants, collagen preparations with impurities are potent immunogens that can stimulate an inflammatory response. Similarly, non-human forms of collagen such as bovine collagen have been associated with a chronic cellular inflammatory reaction that can result in scar tissue and adhesion formation, and transient low-grade fevers. In addition, the duration of implantable collagen is limited, requiring procedures (especially for cosmetic enhancement) to be repeated on a regular basis.

The present invention discloses novel compositions, devices and methods for prolonging the activity of collagen-based implants, and further provides other related advantages.

### SUMMARY OF THE INVENTION

Briefly stated, the present invention provides compositions and methods for prolonging the activity of collagen-based implants. Collagen-based biomaterials are used to provide structure and support in a variety of medical procedures including dermal injections for cosmetic purposes (wrinkles, scars, contour defects), periurethral bulking agents for the management of incontinence, and vascular “plugs” to produce hemostasis following vascular puncture procedures. While extremely effective, collagen implants have a short duration of activity in vivo since the material is rapidly broken down by degradative enzymes (principally collagenase and other matrix metalloproteinase enzymes) released by white blood cells and connective tissue cells (fibroblasts) adjacent to the implant. The result is that the collagen implant procedure must be repeated at frequent intervals to maintain functional activity.

A variety of naturally occurring and synthetically created molecules have been developed for other purposes (e.g., the treatment of malignancy, arthritis and other disorders) that inhibit the activity of collagenase (collectively known as “matrix metalloproteinase inhibitors” or “collagenase inhibitors”). The present invention describes compositions combining collagen and a compound that inhibits the activity of collagenase to produce a collagen-based implant with enhanced durability in vivo (“Collajolie”). Particularly preferred compounds or factors inhibit the activity MMP-1, MMP-8, MMP-13, and/or MMP-14. Representative examples of MMPI suitable for use within the present invention include TIMP-1, tetracycline, doxycycline, minocycline, Batimistate, Marimistat®, RO-1130830, CGS 27023A, BMS-275291, CMT-3, Solimastat, Ilomastat, CP-544439, Prinomastat, PNU-1427690, SU-5402, and Trocade.

Hence, within one aspect of the present invention compositions are provided comprising collagen and a MMPI. Within certain embodiments the MMPI is a Tissue Inhibitor of Matrix Metalloproteinase (e.g., TIMP-1, TIMP-2, TIMP-3, or, TIMP-4). Within other embodiments, the MMPI is tetracycline, or an analog or derivative thereof (e.g., minocycline, or, doxycline); a hydroxamate (e.g., Batimistat, Marimistat, or, Trocade); or RO-1130830, CGS 27023A, CMT-3, Solimastat, Ilomastat, CP-544439, Prinomastat, PNU-1427690, SU-5402, or BMS-275291. Within other embodiments the collagen is a type I or type II collagen. Within yet other embodiments the compositions provided herein may contain other compounds or compositions, including for example, thrombin and/or dyes. Within further embodiments, the composition may be sterilized in a manner suitable for human administration.

Within certain aspects of the present invention, methods are provided for making the compositions described herein, comprising the step of mixing a Collagen and one or more MMPI as described herein. Within related embodiments, such methods can further comprise the step of admixing a dye or a thrombin. Within further embodiments, the composition may be sterilized.

Within other aspects of the present invention, the above-described compositions may be utilized to treat and/or prevent a variety of medical conditions. For example, within one aspect of the present invention methods are provided for the repair and/or augmentation of skin or tissue, comprising injecting into the skin or tissue a composition as described above. Within various embodiments, such compositions may be injected into the lips in order to correct or enhance the lips, or injected into the skin (e.g., into the face in order to correct scars or to diminish wrinkles).

Within other aspects of the present invention, methods are provided for treating or preventing urinary incontinence, comprising administering to a patient a composition as described above. Within certain embodiments, the composition may be administered either periurethrally or transurethrally.

Within yet other aspects of the present invention, methods are provided for sealing a surgical site, comprising the step of administering to a patient a composition as described above. Representative examples of such surgical sites include sites of vascular access (e.g., the sealant can be used as a vascular sealant).

In another aspect, the present invention provides methods of making collajolie, comprising admixing collagen and at least one MMPI. Within particular embodiments, the collajolie is made having at least two MMPI. In certain embodiments, the collagen is type I or type II collagen. In other embodiments, the MMPI is a Tissue Inhibitor of Matrix Metalloproteinase (TIMP), such as TIMP-1, TIMP-2, TIMP-3 or TIMP-4. In yet other embodiments, MMPI is tetracycline, or an analog or derivative thereof such as minocycline or doxycline. In still another embodiment, the MMPI is a hydroxamate such as Batimistat, Marimistat, or, Trocade. Within other embodiments, the MMPI is RO-1130830, CGS-27023A or BMS-275291. Within further embodiments, the MMPI is a polypeptide inhibitor, such as an inhibitor of a metalloprotease maturase. In additional embodiments, the MMPI is a mercapto-based compound. Within other embodiments, the MMPI is a bisphosphonate with structure (I):

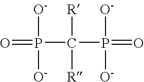

wherein R′ and R″ are independently a hydrogen, a halogen such as chlorine, a hydroxy, an optionally substituted amino group, an optionally substituted thio group, or an optionally substituted alkyl, alkanyl, alkenyl, alkynyl, alkyldiyl, alkyleno, heteroalkyl, heteroalkanyl, heteroalkenyl, heteroalkanyl, heteroalkyldiyl, heteroalkyleno, aryl, arylalkyl, heteroaryl, heteroarylalkyl. Within other embodiments, prior to admixture with collagen, said MMPI is first admixed with at least one polymer. In a related embodiments, the polymer is biodegradable, such as albumin, gelatin, starch, cellulose, dextrans, polysaccharides, fibrinogen, poly (esters), poly (D,L lactide), poly (D,L-lactide-co-glycolide), poly (glycolide), poly(e-caprolactone), poly (hydroxybutyrate), poly (alkylcarbonate), poly(anhydrides), or poly (orthoesters), and copolymers and blends thereof. In another embodiment, any of the aforementioned methods of making collajolie further comprise the step of sterilizing said mixture.

These and other aspects of the present invention will become evident upon reference to the following detailed description and attached drawings. In addition, various references are set forth herein which describe in more detail certain procedures or compositions (e.g., compounds, proteins, vectors, and their generation, etc.), and are therefore incorporated by reference in their entirety. When PCT applications are referred to it is also understood that the underlying or cited U.S. applications are also incorporated by reference herein in their entirety.

### DETAILED DESCRIPTION OF THE INVENTION

Prior to setting forth the invention, it may be helpful to an understanding thereof to set forth definitions of certain terms that will be used hereinafter.

“Collagen” as used herein refers to all forms of collagen as are described or referenced herein, including those that have been processed or modified. Representative examples include type I and type II collagen. Collagen may be prepared from human or animal sources, or, may be produced using recombinant techniques.

“Matrix Metalloproteinase Inhibitor” or “MMPI” refers to a compound, agent or composition that inhibits matrix metalloproteinase activity. Representative examples of MMP Inhibitors (“MMPI”) include Tissue Inhibitors of Metalloproteinases (TIMPs) (e.g., TIMP-1, TIMP-2, TIMP-3, or TIMP-4), α₂-macroglobulin, tetracyclines (e.g., tetracycline, minocycline, and doxycycline), hydroxamates (e.g., Batimistat, Marimistat and Trocade), chelators (e.g., EDTA, Cysteine, Acetylcysteine, D-penicillamine, and gold salts), synthetic MMP fragments, succinyl mercaptopurines, phosphonamidates, and hydroxaminic acids.

Any concentration or percentage ranges recited herein are to be understood to include concentrations of any integer within the range and fractions thereof, such as one tenth and one hundredth of an integer, unless otherwise indicated. As used herein, “about” or “comprising essentially of” means±15%.

I. Collagen

Collagen is the major component in skin, cartilage, bone, and connective tissue, and occurs in several different types or forms, with Types I, II, III, and IV being most common. Collagen has typically been isolated from natural sources, such as bovine bone, cartilage, or, hide. Bones are usually defatted, crushed, dried, and demineralized to extract the collagen. In contrast, bovine cartilage or hide is usually minced and digested with enzymes other than collagenase (in order to remove contaminating protein). Collagen can also be prepared from human tissue (the patient's own or donor tissue) or by recombinant methods.

Within certain embodiments of the invention, preferred collagens are prepared as non-immunoreactive sterile compositions. They may be soluble (e.g., commercially available Vitrogen® 100 collagen-in-solution), or be in the form of reconstituted fibrillar atelopeptide collagen, for example Zyderm®(Collagen Implant (ZCI).

Representative examples of patents which disclose collagen-containing compositions, devices, and methods for making and/or delivering such compositions and devices include U.S. Pat. Nos. 4,164,559, 4,424,208, 4,140,537, 4,563,350, 4,582,640, 4,642,117, 4,743,229, 4,776,890, 4,795,467, 4,888,366, 5,035,715, 5,162,430, 5,304,595, 5,324,775, 5,328,955, 5,413,791, 5,428,022, 5,446,091, 5,475,052, 5,523,348, 5,527,856, 5,543,441, 5,550,187, 5,565,519, 5,580,923, 5,614,587, 5,616,689, 5,643,464, 5,693,341, 5,744,545, 5,752,974, 5,756,678, 5,786,421, 5,800,541, 5,807,581, 5,823,671, 5,874,500, 5,895,833, 5,936,035, 5,962,648, 6,090,996, 6,096,039, 6,111,165, 6,165,489, 6,166,130, 6,280,727, 6,312,725, and 6,323,278.

II. Matrix Metalloproteinase (MMP) Inhibitors

Metalloproteinases (MMPs) are a group of naturally occurring zinc-dependent enzymes involved in the breakdown and turnover of extracellular matrix macromolecules. Over 23 metalloproteinases have been identified to date and have been broadly categorized into families of enzymes known as collagenases, stromelysins, gelatinases, elastases and matrilysins. Metalloproteinases are derived from a variety of cell types including neutrophils, monocytes, macrophages and fibroblasts.

MMPs are the principle enzymes involved in the breakdown and normal turnover of collagen in vivo. Although numerous MMPs are capable of breaking down several connective tissue elements including collagen, the enzymes with the highest specificity for collagen come from the collagenase family (e.g., MMP-1, MMP-8, MMP-13 and MMP-14). Metalloproteinase activity is inhibited naturally in vivo by a family of inhibitors known as “Tissue Inhibitors of Metalloproteinase” or “TIMPs” which bind to the active region of the metalloproteinase enzyme rendering it inactive. It is the natural balance between enzyme activity and inhibition that regulates the rate of metabolism of the extracellular matrix under physiologic conditions.

Assays for measuring MMP inhibition are readily known in the art, and include, for example, the following: Cawston T. E., Barrett A. J., “A rapid and reproducible assay for collagenase using [14C] acetylated collagen,” Anal. Biochem. 35:1961-1965 (1963); Cawston T. E., Murphy G., “Mammalian collagenases,” Methods in Enzymology 80:711 (1981); Koshy P. T. J., Rowan A. D., Life P. F., Cawston T. E., “96-well plate assays for measuring collagenase activity using (3)H-acetylated collagen,” Anal. Biochem. 99:340-345 (1979); Stack M. S., Gray R. D., “Comparison of vertebrate collagenase and gelatinase using a new fluorogenic substrate peptide,” J. Biol. Chem. 264:4277-4281 (1989); and Knight C. G, Willenbrock F., Murphy G., “A novel coumarin-labelled peptide for sensitive continuous assays of the matrix metalloproteinases,” FEBS Lett 296:263-266 (1992). Within the context of this invention, an MMPI would preferably have a Inhibitory Concentration (IC) ranging from mM to nM (10-3 to 10-9).

When collagen is implanted as part of a therapeutic procedure, it too is gradually metabolized by enzymes from the MMP family until it is fully resorbed. This gradual loss of structural integrity due to enzymatic degradation of the collagen implant results in loss of functional activity leading to implant failure and, ultimately, the need for subsequent reintervention. Attempts at prolonging the activity of the collagen implant have centered on crosslinking the collagen implant so as to slow enzymatic degradation. The present invention describes incorporating into the collagen implant an agent or agents capable of inhibiting MMP activity so as to tip the physiologic balance in favor of collagen preservation. This invention is compatible with, and can be used in combination with strategies, such as collagen crosslinking, designed to increase the residence time of a collagen implant.

Since pathologic production of MMPs has been associated with a variety of clinically important disease processes such as tumor metastasis and the progression of chronic inflammatory conditions such as osteoarthritis and rheumatoid arthritis, numerous naturally occurring and synthetic agents have been developed to inhibit MMP activity. Not surprisingly, regulation of MMP activity is an important and highly regulated process in vivo. As a result there are numerous sites in the pathway leading to MMP production where it is possible to develop molecules capable of inhibiting MMP synthesis or activity. The types of agents capable of inhibiting MMP activity are described in more detail below.

Briefly, a variety of cytokines (e.g., TNF-α, IL-1, FGF and others) are capable of stimulating the pathway which leads to the production of MMPs. Inhibitors of these cytokines or agents which block their cellular receptors have been demonstrated to inhibit MMP synthesis under certain circumstances and would be suitable for use this invention. After binding to its cellular receptor, the stimulus for MMP production triggers the production of a variety of second messengers and cell signaling molecules (e.g., jun kinase, JKK, etc.)—inhibition of these molecules can also reduce the production of MMPs. A variety of transcription factors (e.g., c-fos, c-jun, NFκ-B, c-myc) have been implicated in the transcription of the MMP genes. Inhibitors of these transcription factors and their products (e.g., the AP-1 protein) can also decrease the amount of MMPs transcribed and can be utilized for the purposes of this invention. Similarly, strategies that inhibit the MMP gene itself (e.g., gene knockout) or MMP RNA (e.g., antisense, ribozymes, tetracycline, doxycycline, minocycline) can be utilized in this invention to decrease the amount of active MMP enzyme in the region surrounding the collagen implant.

In addition, it is possible to inhibit the function and activity of metalloproteinases after they have been secreted from the cell. Since MMPs are secreted from the cell as inactive precursor proteins (called Pro-MMPs) that are subsequently converted to the active enzyme through a highly specific enzymatic cleavage (catalyzed by enzymes such as plasmin, mast cell protease, cathepsin G, plasma kallikrein and others), it is possible to inhibit the conversion of the MMP from its inactive to active state (thereby maintaining it in an inactive form). Inhibitors of the enzymes responsible for the conversion of the MMP from its inactive to active state can also be utilized for this invention. And lastly, it is possible to directly inhibit the function of an activated MMP through several mechanisms such as chelation of its zinc metal active center (e.g., EDTA, Cysteine, Acetylcysteine, D-penicillamine, Gold Salts; hydroxamates such as Batimistat, Marimistat, Trocade, Actinonin, Matylystatins; phosphonic acid inhibitors; phosphonates; phosphonamidates; thiols and sulfodiimines which form monodentate coordination with the catalytic zinc; carboxylates which form bidentate coordination with the catalytic zinc; succinyl mercaptoketones and mercaptoalcohols). These compounds are quite effective at inhibiting MMP activity and would be particularly useful for the purposes of this invention. An important class of MMPIs exert their effect through specific binding to the MMP leading to the formation of an inactive complex. These compounds, known as Tissue Inhibitors of Metalloproteinases (TIMPs) such as TIMP-1, TIMP-2, TIMP-3, and TIMP-4, are capable of inhibiting the activity of virtually all of the MMPs. Although any of the TIMPs would be suitable for the purposes of this invention, TIMP-1 (and to a lesser extent TIMP-2) would be particularly preferred as it as the highest specificity for inhibition of collagenase. It should also be noted that any compound which increased the production of TIMPs would tip the balance in favor of collagen preservation and could be of utility in this invention. Still other inhibitors act by preventing binding of the MMP to its substrate (e.g., Synthetic MMP Fragments, Synthethetic Collagen Fragments) and could also be utilized alone, or in combination with other MMPIs for the purpose of this invention). It should be clear to one of skill in the art that regardless of the specific mechanism of inhibition, any agent capable of inhibiting the production, activation or enzymatic function of the MMP enzymes would be ideal agents for the purposes of this invention.

Representative examples of MMPIs include actinonin (3-[[1-[[2-(hydroxymethyl)-1-pyrolidinyl]carbamoyl]-octano-hydroxamic acid); bromocyclic-adenosine monophosphate; N-chlorotaurine; batimastat also known as BB-94; CT1166, also known as N1 {N-[2-(morpholinosulphonylamino)-ethyl]-3-cyclohexyl-2-(S)-propanamidyl}-N-4-hydroxy-2-(R)-[3-(4-methylphenyl)propyl]-succinamide (Biochem. J. 308:167-175 (1995)); estramustine (estradiol-3-bis(2-chloroethyl)carbamate); eicosa-pentaenoic acid; marimastat (BB-2516); matlystatin-B; peptidyl hydroxamic acids such as pNH₂-Bz-Gly-Pro-D-Leu-D-Ala-NHOH (Biophys. Biochem. Res. Comm. 199:1442-1446 (1994)); N-phosphonalkyl dipeptides such as N-[N-((R)-1-phosphonopropyl)-(S)-leucyl]-(S)-phenylalanine-N-methylamide (J. Med. Chem. 37:158-169 (1994)); protocatechuic aldehyde (3,4-dihydroxybenzaldehyde); Ro-31-7467, also known as 2-[(5-bromo-2,3-dihydro-6-hydroxy-1,3-dioxo-1 Hbenz[de]isoquinolin-2-yl)methyl](hydroxy)-[phosphinyl]-N-(2-oxo-3-azacyclotridecanyl)-4-methylvaleramide; tetracyclines such as (4-(dimethylamino)-1,4,4a,5,5a,6,11,12a-octahydro-3,6,10,12,12a-pentahydroxy-6-methyl-1,11-dioxo-2-naphthacenecarboxamide), doxycycline (α-6-deoxy-5-hydroxy-tetracycline) minocycline (7-dimethylamino-6-dimethyl-6-deoxytetracycline), and methacycline (6-methylene oxytetracycline); trifluoroacetate (J Med Chem. 36:4030-4039 (1993)); and 1,10-phenanthroline (o-phenanthroline [4-(N-hydroxyamino)-2R-isobutyl-3 S-(thiopen-2-ylthiomethyl)-succinyl]-L-phenylalanine-N-methylamidecarboxyalkylamino-based compounds such as N-[1-(R)-carboxy-3-(1,3-dihydro-2H-benz[f]isoindol-2-yl)propyl]-N′,N′-dimethyl-L-leucinamide.

Other representative MMPIs include, for example, chelators (e.g., EDTA, Cysteine, Acetylcysteine, D-penicillamine, and Gold Salts), bis(dioxopiperzaine, (see U.S. Pat. No. 5,866,570, to Liang et al.,), neovastat (inhibits gelatinolytic and elastinolytic activities for MMP-2, MMP-9, and MMP-12, see U.S. Pat. No. 6,168,807 Aetema Laboratorie); KB-R7785 (Akzo Nobel); Ilomastat (Glycomed/Ligand; U.S. Pat. No. 5,892,112); RPR-122818 (Aventis); solimastat (British Biotech, WO 99/25693), BB-1101, BB-2983, BB-3644 (British Biotech); BMS-275291 (see Rizvi et al., Proceedings of the 1999 AACR NCI EORTC International Conference “#726 “A Phase I, safety and pharmacokinetic trial of BMS-275291, a matrix metalooproteinase inhibitor (MMPI), in patients with advanced or metastatic cancer”), D-1927, D-5410, CH-5902, CH-138 (Celltech); CMT-3, dermostat (CollaGenex—U.S. Pat. No. 5,837,696); DAC-MMPI (ConjuChem); RS-1130830 and RS-113-080 (Hoffmann-La Roche); GM-1339 (Ligand); GI-155704A (GlaxoSmith Kline); ONO-4817 (ONO); AG-3433, AG-3088, prinomastat (Agouron; U.S. Pat. No. 5,753,653), CP-544439 (Pfizer; U.S. Pat. No. 6,156,798); POL-641 (Polifarma); SC-964, SD-2590, PNU-142769 (Pharmacia; WO 97/32846), SU-5402 (Pharmacia; WO 98/50356); PGE-2946979, PGE-4304887 (Procter & Gamble); fibrolase-conjugate (Schering-AG); EF-13 (Scotia-Pharmaceuticals); S-3304 (Shionogi); CGS-25015 and CGS-27023A (Novartis), XR-168 (Xenova), and RO 1130830 (Fisher et al., 219 American Chemical Society National Meeting, San Francisco, Calif., March 26-30, 2000, “ORGN 830 “Synthesis of RO 1130830, a Matrix Metalloproteinase Inhibitor: Evolution of a Research Scheme to Pilot-Plant Production”). Other MMPIs are described in U.S. Pat. Nos. 4,235,885; 4,263,293; 4,276,284; 4,297,275; 4,367,233; 4,371,465; 4,371,466; 4,374,765; 4,382,081; 4,558,034; 4,704,383; 4,950,755; 5,270,447, 6,294,694, and 6,329,550.

Representative examples of classes of MMPIs which are discussed in more detail below include (1) Tissue Inhibitors of Matrix Metalloproteinases (TIMPs); (2) tetracyclines, (3) hydroxamates, (4) synthetic MMP fragments (e.g., peptide inhibitors), (5) mercapto-based compounds, and (6) bisphosphonates.

1. Tissue Inhibitors of Matrix Metalloproteinase

Tissue Inhibitors of Matrix Metalloproteinases (TIMPs) are classified based upon their ability to inhibit metalloproteinases, structural similarity to each other, the 12 cysteines which form disulfide bonds important in secondary structure, and the presence of a VIRAF motif which interacts with the metal ion of the metalloproteinases. The nucleic acid and amino acid sequences of TIMPs have been described: TIMP-1 (Docherty A J P et al. (1985) Nature 318: 66-69), TIMP-2 (Boone T C et al. (1990) Proc Natl Acad Sci 87: 2800-2804; Stetler-Stevenson W G et al. (1990) J Biol Chem 265: 13933-38), and TIMP-3 (Wilde C G et al. (1994) DNA Cell Biol 13: 711-18; Apte et al., “The Gene Structure of Tissue Inhibitor of Metalloproteinases” (TIMP)-3 and Its Inhibitory Activities Define the Distinct TIMP Gene Family); (See also, Boone T. C., et al., “cDNA cloning and expression of a metalloproteinase inhibitor related to tissue inhibitor of metalloproteinases,” Proc. Natl. Acad. Sci. USA, 87:2800-2804 (April 1990), Freudenstein, mRNA of bovine tissue inhibitor of metalloproteinase: Sequence and expression in bovine ovarian tissue, Biochem Biophys. Res. Comm., 171:250-256(1990), U.S. Pat. Nos. 5,643,752 and 6,300,310).

TIMP-1 is a 30 kD protein, and is the most commonly expressed TIMP molecule. It contains two asparagine residues which act as carbohydrate binding sites, one in loop 1 and one in loop 2 (Murphy and Docherty, supra). In addition, a truncated form of TIMP-1 which contains only the first three loops of the molecule is able to inhibit MMPs. Although TIMP-1 is a better inhibitor of interstitial collagenase than TIMP-2 (Howard E W et al. (1991) J Biol Chem 266: 13070-75), the 23 kD TIMP-2 molecule is the most effective inhibitor of gelatinases A and B. TIMP-3 is a 21 kD protein which inhibits collagenase 1, stromelysin, and gelatinases A and B (Apte S. S. et al. (1995) J Biol Chem 270: 14313-18) and may be induced by mitogens (Wick et al. (1994) J Biol Chem 269: 18953-60).

As described above, any of the four TIMP molecules are capable of inhibiting the activity of virtually all of the MMPs identified to date and would be suitable for the purposes of this invention. However, TIMP-1, which has a high specificity for the inhibition of collagenase, would be particularly preferred for incorporation into a collagen implant.

2. Tetracyclines

Tetracyclines are a class of analog and derivative compounds known originally for their use as antibiotics. Numerous tetracyclines, including tetracycline, doxycycline, minocycline and others, have been demonstrated to inhibit the production and activity of MMPs. Although the exact mechanism is incompletely understood, MMP inhibition may occur through downregulation of MMP expression and/or post-translationally through chelation of the zinc metal active site. Given their widespread use and low toxicity, these compounds would be of particular utility for incorporation into a collagen implant.

The parent compound of the tetracycline family, tetracycline, has the following general structure:

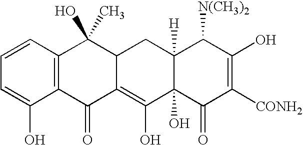

The multiple ring nucleus can be numbered as follows:

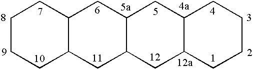

Tetracycline, as well as the 5-OH (oxytetracycline) and 7-Cl (chlorotetracycline) derivatives exist in nature and are well known antibiotics. Other tetracyclines include, for example, apicycline, chelocardin, clomocycline, demeclocycline, doxycycline, etamocycline, guamecycline, lymecycline, meglucyccline, mepycyhcline, minocycline, methacycline, penimepicycline, piacycline, rolitetracycline, and sancycline.

Tetracycylines can also be modified so that they retain their structural relationship to antibiotic tetracyclines, but have their antibiotic activity substantially or completely reduced by chemical modification. Representative examples of chemically modified tetracyclines (CMT's) include, for example, CMT-1 (4-de(dimethylamino)-tetracycline), CMT-2 (tetracyclinonitrile), CMT-3 (6-demethyl-6-deoxy-4-de(dimethylamino)tetracycline), CMT-4 (7-chloro-4-de(dimethylamino)tetra-cycline), CMT-5 (tetracycline pyrazole), CMT-6 (4-hydroxy-4-de(dimethylamino)tetra-cycline), CMT-7 (4-de(dimethylamino)-12α-deoxytetracycline), CMT-8 (6-deoxy-5α-hydroxy-4-de(dimethylamino)tetracycline), CMT-9 (4-de(dimethylamino)-12α-deoxyanhydro-tetracycline), and CMT-10 (4-de(dimethylamino)minocycline).

Representative examples of tetracyclines (including tetracycline derivatives) are described in U.S. Pat. No. 3,622,627 to Blackwood et al., U.S. Pat. No. 3,846,486 to Marcus, U.S. Pat. No. 3,862,225 to Conover et al., U.S. Pat. No. 3,895,033 to Murakami et al., U.S. Pat. No. 3,901,942, to Bernardi et al., U.S. Pat. No. 3,914,299 to Muxfeldt, U.S. Pat. No. 3,925,432 to Gillchriest, U.S. Pat. No. 3,927,094 to Villax, U.S. Pat. No. 3,932,490 to Fernandez, U.S. Pat. No. 3,951,962 to Murakami et al., U.S. Pat. No. 3,983,173 to Hartung et al., U.S. Pat. No. 3,991,111 to Murakami et al., U.S. Pat. No. 3,993,694 to Martin et al., U.S. Pat. No. 4,060,605 to Cotti, U.S. Pat. No. 4,066,694 to Blackwood et al., U.S. Pat. No. 4,081,528 to Armstrong, U.S. Pat. No. 4,086,332 to Armstrong, U.S. Pat. No. 4,126,680 to Armstrong, U.S. Pat. No. 4,853,375 to Krupin et al., U.S. Pat. No. 4,918,208 to Hasegawa et al., and U.S. Pat. No. 5,538,954 to Koch et al. (see generally, Mitscher, L. A., The Chemistry of Tetracycline Antibiotics, ch. 6, Marcell Dekker, New York, 1978).

Further examples of tetracycline derivatives are disclosed in U.S. Pat. No. 4,666,897 to Golub et al., U.S. Pat. No. 4,704,383 to McNamara et al., U.S. Pat. No. 4,904,647 to Kulcsar et al., U.S. Pat. No. 4,935,412 to McNamara et al., U.S. Pat. No. 5,223,248 to McNamara et al., U.S. Pat. No. 5,248,797 to Sum et al., U.S. Pat. No. 5,281,628 to Hlavka et al., U.S. Pat. No. 5,326,759 to Hlavka et al., U.S. Pat. No. 5,258,371 to Golub et al., U.S. Pat. No. 5,308,839 to Golub et al., U.S. Pat. No. 5,321,017 to Golub et al., U.S. Pat. No. 5,326,759 to U.S. Pat. No. 5,401,863 to Hlavka et al., U.S. Pat. No. 5,459,135 to Golub et al., U.S. Pat. No. 5,530,117 to Hlvaka et al., U.S. Pat. No. 5,563,130 to Backer et al., U.S. Pat. No. 5,567,693 to Backer et al., U.S. Pat. No. 5,574,026 to Backer et al., U.S. Pat. No. 5,698,542 to Zheng et al., U.S. Pat. No. 5,773,430 to Simon et al., U.S. Pat. No. 5,834,450 to Su, U.S. Pat. No. 5,843,925 to Backer et al., U.S. Pat. No. 5,856,315 to Backer et al., U.S. Pat. No. 6,028,207 to Zheng et al., U.S. Pat. No. 6,143,161 to Heggie et al. and U.S. Pat. No. 6,165,999 to Vu, as well as PCT publication Nos. WO 99/33455, WO 99/37306, WO 99/37307, WO 00/18353 and WO 00/28983.

3. Hydroxamates

A further class of compounds which inhibit MMPs are hydroxamates (or hydroxamic acids). Although the exact mechanism of MMP inhibition is not precisely known, it is believed these compounds exert their effect primarily through interaction with the zinc metal active site in the enzyme (e.g., by coordinating with the catalytic zinc in a bidentate manner to adopt a triagonal bipyrimidal geometry). A variety of hydroxamates have been synthesized and tested in several disease states with mixed clinical results. However, given their selective activity against MMPs and their excellent safety and tolerability, these agents would be particularly preferred for incorporation into a collagen implant to enhance the durability of the implant.

Hydroxamates (or hydroxamic acids) have the general structures shown below:

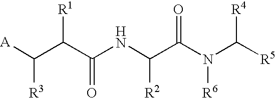

wherein A is HN(OH)—CO— or HCO—N(OH)—; R¹ is C₂-C₅ alkyl; R² is the characterizing group of a natural a amino acid which may be protected provided that R² is not H or methyl; R³ is H, NH₂, OH, SH, C₁-C₆ alkyl, C₁-C₆ alkoxy, C₁-C₆ alkylamino, C₁-C₆ alkylthio, aryl (C₁-C₆ alkyl), or amino(C₁-C₆ alkyl), hydroxy(C₁-C₆ alkyl), mercapto(C₁-C₆ alkyl) or carboxy(C₁-C₆ alkyl) where the amino, hydroxy, mercapto or carboxyl group can be protected, the amino group may be acylated or the carboxyl group may be amidated; R⁴ is H or methyl; R⁵ is H, C₁-C₆ alkyl, C₁-C₆ alkoxy(C₁-C₆ alkyl), di(C₁-C₆ alkoxy)methylene, carboxy, (C₁-C₆ alkyl)carbonyl, (C₁-C₆ alkoxy)carbonyl, arylmethoxycarbonyl, (C₁-C₆ alkyl)aminocarbonyl or arylaminocarbonyl; and R⁶ is H or methyl; or R² and R⁴ together form a group (CH₂)ₙ where n is an integer from 4 to 11; or R⁴ and R⁵ together form a trimethylene group, and pharmaceutically acceptable salts of these hydroxymate compounds that are either acidic or basic. In this regard, see, e.g., EP-A-0236872.

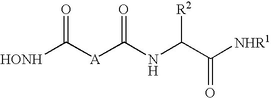

wherein R¹ is C₁-C₆ alkyl; R2 is C₁-C₆ alkyl, benzyl, hydroxybenzyl, benzyloxybenzyl, (C₁-C₆ alkoxy) benzyl or benzyloxy (C₁-C₆ alkyl); A is a (CHR³—CHR⁴) or (CR³═CR⁴) group; R³ is hydrogen, C₁-C₆ alkyl, phenyl or phenyl (C₁-C₆ alkyl); and R⁴ is H or C₁-C₆ alkyl, phenyl (C₁-C₆ alkyl), cycloalkyl or cycloalkyl (C₁-C₆ alkyl). In this regard, see, e.g., EP-A-0214639.

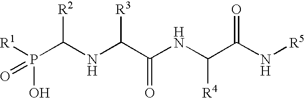

wherein R¹ is hydrogen or hydroxy, R² is hydrogen or alkyl, R³ is C₃-C₆ alkyl, R⁴ is hydrogen, alkyl, —CH₂Z where Z is optionally substituted phenyl or heteroaryl, or R⁴ is a group C(HOR⁸)R⁹ where R⁸ is hydrogen, alkyl of CH₂Ph where Ph is optionally substituted phenyl, and R⁹ is hydrogen or alkyl; and R⁵ is hydrogen or alkyl. In this regard, see, e.g., EP-A-320118.

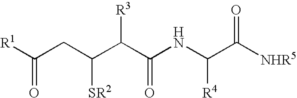

wherein R¹ is hydrogen, alkyl or optionally substituted aryl, R² is hydrogen or acyl such as CO alkyl or COZ where Z is optionally substituted aryl; R³ is C₃₋₆ alkyl, R⁴ is hydrogen, alkyl, —CH₂R¹⁰ where R¹⁰ is optionally substituted phenyl or heteroaryl, or R⁴ is a group C(HOR¹¹)R¹² where R¹¹ is hydrogen, alkyl or CH₂Ph where Ph is optionally substituted phenyl, and R¹² is hydrogen or alkyl; and R⁵ is hydrogen, alkyl or a group C(HR¹³)COR¹⁴ where R¹³ is hydrogen, or alkyl, and R¹⁴ is hydroxy, alkoxy, or —NR⁶R⁷, where each of R⁶ or R⁷ is hydrogen or alkyl, or R⁶ and R⁷ together with the nitrogen atom to which they are bonded form a 5-, 6 or 7 membered ring with optional oxygen or sulfur atom in the ring or an optional further nitrogen atom optionally substituted by alkyl. In this regard, see, e.g., EP-A-0322184.

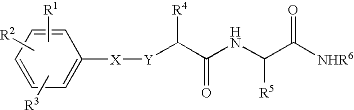

wherein R¹ and R² are independently H, alkyl, alkoxy, halogen or CF₃, R³ is H, acyl, such as COalkyl or COZ, where Z is optionally substituted aryl, or a group RS where R is an organic residue such that the group RS provides an in vivo cleavable disulphide bond; R⁴ is C₃-C₆ alkyl, R⁵ is H, alkyl, —CH₂R¹⁰ where R¹⁰ is optionally substituted phenyl or heteroaryl, or a group C(HOR¹¹))R¹² where R¹¹ is hydrogen, alkyl or CH₂Ph where Ph is optionally substituted phenyl, and R¹² is hydrogen or alkyl; and R⁶ is hydrogen, alkyl or a group C(HR¹³)COR¹⁴ where R¹³ is hydrogen, or alkyl, and R¹⁴ is hydroxy, alkoxy, or —NR⁷R⁸, where each of R⁷ or R⁸ is hydrogen or alkyl, or R⁷ and R⁸ together with the nitrogen atom to which they are bonded form a 5-, 6- or 7-membered ring with optional oxygen, sulphur or optionally substituted nitrogen atom in the ring; or R⁵ and R⁶ are joined together as (CH₂)ₘ where m is an integer from 4 to 12; X is (CH₂)ₙ where n is 0, 1, or 2; and Y is CH₂. In this regard, see, e.g., EP-A-358305.

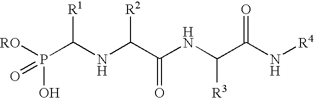

wherein R is hydrogen, C₁-C₆ alkyl or optionally substituted benzyl, R¹ is hydrogen or C₁-C₆ alkyl, R² is C₃-C₆ alkyl, R³ is hydrogen, alkyl, —CH₂Z where Z is optionally substituted phenyl or heteroaryl, or R³ is a group C(HOR⁷)R⁸ where R⁷ is hydrogen, alkyl or CH₂Ph where Ph is optionally substituted phenyl, and R⁸ is hydrogen or alkyl; and R⁴ is —CH₂—(CH₂)ₙOR⁵, —CH₂—(CH₂)ₙOCOR⁶ or —CH(R⁹)COR¹⁰, where n is an integer from 1 to 6; R⁵, R⁶ and R⁹ are hydrogen or C₁-C₆ alkyl; and R10 is hydroxy or O(C₁-C₆ alkyl) or NR⁵R⁶ where R⁵ and R⁶ may be linked to form a heterocyclic ring; or R³ and R⁴ are joined together as (CH₂)ₘ where m is an integer from 4 to 12. In this regard, see, e.g., EP-A-0401963.

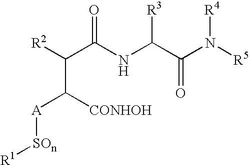

wherein R¹ is H, C₁-C₆ alkyl, phenyl, thienyl, substituted phenyl, phenyl (C₁-C₆)alkyl, heterocyclyl, (C₁-C₆)alkylcarbonyl, phenacyl or substituted phenacyl group; or, when n is 0, R¹ represents SR^{x}, wherein R^{x} represents a group of the formula:

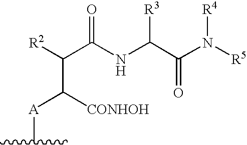

and R² is H, C₁-C₆ alkyl, C₁-C₆ alkenyl, phenyl (C₁-C₆) alkyl, cycloalkyl (C₁-C₆) alkyl or cycloalkenyl (C₁-C₆) alkyl group; R³ is an amino acid side chain or a C₁-C₆ alkyl, benzyl, (C₁-C₆ alkoxy) benzyl, benzyloxy (C₁-C₆ alkyl) or benzyloxybenzyl group; R⁴ is H or a C₁-C₆ alkyl group; R⁵ is H or a methyl group; n is 0, 1 or 2; and A represents a C₁-C₆ hydrocarbon chain, optionally substituted with one or more C₁-C₆ alkyl, phenyl or substituted phenyl groups; and their salts and N-oxides. In this regard, see, e.g., PCT International Publication No. WO90/05719.

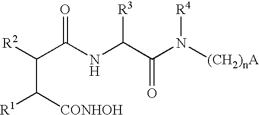

wherein R¹ is H, C₁-C₆ alkyl, C₂-C₆ alkenyl, phenyl, phenyl (C₁-C₆) alkyl, C₁-C₆ alkylthiomethyl, phenylthiomethyl, substituted phenylthiomethyl, phenyl (C₁-C₆) alkylthiomethyl, or heterocyclylthiomethyl or R1 represents —SR^{x} wherein R^{x} represents a group

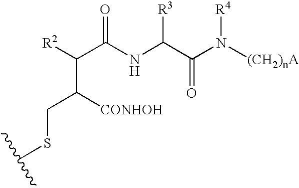

and R² represents a hydrogen atom, or a C₁-C₆ alkyl, C₁-C₆ alkenyl, phenyl (C₁-C₆) alkyl, cycloalkyl (C₁-C₆) alkyl, or cycloalkenyl (C₁-C₆) alkyl; R³ represents an amino acid side chain or a C₁-C₆ alkyl, benzyl, (C₁-C₆) alkoxybenzyl, benzyloxy (C₁-C₆) alkyl, or benzyloxybenzyl group; R⁴ represents a hydrogen atom, or a methyl group; n is an integer from 1 to 6; and A represents the group —NH₂, a substituted acyclic amine or a heterocyclic base; or a salt and/or N-oxide and/or (where the compound is a thio-compound) a sulphoxide or sulphone thereof. In this regard, see, e.g., PCT International Publication No. WO09/05716.

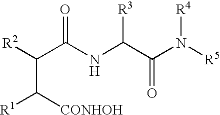

wherein R¹ is H, C₁-C₆ alkyl, C₁-C₆ alkenyl, phenyl, phenyl (C₁-C₆) alkyl, C₁-C₆ alkylthiomethyl, phenylthiomethyl, substituted phenylthiomethyl, phenyl (C₁-C₆) alkylthiomethyl or heterocyclylthiomethyl group; or R1 represents —S—R^{x}, wherein R^{x} represents a group

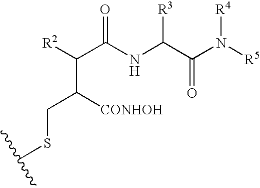

and R2 represents a hydrogen atom, or a C₁-C₆ alkyl, C₁-C₆ alkenyl, phenyl (C₁-C₆) alkyl, cycloalkyl (C₁-C₆) alkyl, or cycloalkenyl (C₁-C₆) alkyl; R³ represents an amino acid side chain or a C₁-C₆ alkyl, benzyl, (C₁-C₆) alkoxybenzyl, benzyloxy (C₁-C₆) alkyl or benzyloxybenzyl group; R⁴ represents a hydrogen atom or a methyl group; R⁵ represents a group (CH₂)ₙA; or R⁴ and R⁵ together represent a group

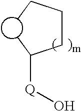

and Q represents CH₂ or CO; m is an integer from 1 to 3; n is an integer from 1 to 6; and A represents a hydroxy, (C₁-C₆) alkoxy, (C₂-C₇) acyloxy, (C₁-C₆) alkylthio, phenylthio, (C₂-C₇) acylamino or N-pyrrolidone group; or a salt and/or N-oxide and/or (where the compound is a thio-compound) a sulphoxide or sulphone thereof. In this regard, see, e.g., PCT International Publication No. WO91/02716.

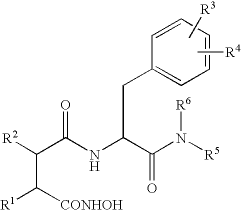

wherein R¹ is H, C₁-C₆ alkyl, phenyl, substituted phenyl, phenyl (C₁-C₆ alkyl), or heterocyclyl; or R¹ is ASOₙR⁷ wherein A represents a C₁-C₆ hydrocarbon chain, optionally substituted with one or more C₁-C₆ alkyl, phenyl or substituted phenyl groups, n is 0, 1, or 2, and R⁷ is C₁-C₆ alkyl, phenyl, substituted phenyl, phenyl (C₁-C₆ alkyl), heterocyclyl, (C₁-C₆ alkyl) acyl, thienyl or phenacyl; R2 is hydrogen, C₁-C₆ alkyl, C₁-C₆ alkenyl, phenyl (C₁-C₆ alkyl) or cycloalkyl (C₁-C₆ alkyl); R³ and R⁴ are selected from hydrogen, halogen, cyano amino, amino (C₁-C₆) alkyl, amino di (C₁-C₆) alkyl, amino (C₁-C₆) alkylacyl, aminophenacyl, amino (substituted) phenacyl, amino acid or derivative thereof, hydroxy, oxy (C₁-C₆) alkyl, oxyacyl, formyl, carboxylic acid, carboxamide, carboxy (C₁-C₆) alkylamide, carboxyphenylamide, carboxy (C₁-C₆) alkyl, hydroxy (C₁-C₆) alkyl, (C₁-C₆) alkyloxy (C₁-C₆) alkyl or acyloxy (C₁-C₆) alkyl, (C₁-C₆) alkylcarboxylic acid, or (C₁-C₆) alkylcarboxy (C₁-C₆) alkyl; or R³ is OCH₂COR⁸ and R⁴ is hydrogen wherein R⁸ is hydroxyl, C₁-C₆ oxyalkyl, C₁-C₆ oxyalkylphenyl, amino, C₁-C₆ aminoalkyl, C₁-C₆ aminodialkyl, C₁-C₆ aminoalkylphenyl, an amino acid or derivative thereof; or R³ is OCH₂CH₂OR⁹ and R⁴ is hydrogen wherein R⁹ is C₁-C₆ alkyl, C₁-C₆ alkylphenyl, phenyl, substituted phenyl, (C₁-C₆ alkyl)acyl, or phenacyl; or R3 is OCH₂CN and R4 is hydrogen; R⁵ is hydrogen or C₁-C₆ alkyl, or (C₁-C₆) alkylphenyl; R⁶ is hydrogen or methyl; or a salt thereof. In this regard, see, e.g., PCT International Application No. PCT/GB92/00230.

Two preferred compounds for use in the present invention, which are mentioned in U.S. Pat. No. 5,872,152, are: [4-(N-hydroxyamino)-2R-isobutyl-3S-thienylthiomethyl)succinyl]-L-phenylalanine-N-methylamide, having the structure below

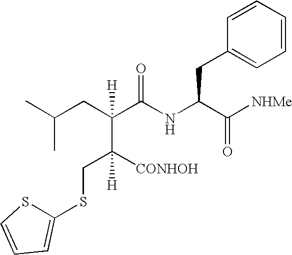

and [4-(N-hydroxyamino)-2R-isobutyl-3 S-phenylthiomethyl)succinyl]-L-phenylalanine-N-methylamide, having the structure below

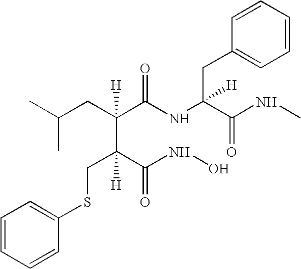

As used herein for describing MMP inhibitors having a hydroxamic acid moiety, the following terms have the indicated meanings. The term “C₁-C₆ alkyl” refers to straight chain or branched chain hydrocarbon groups having from one to six carbon atoms, where illustrative alkyl groups are methyl, ethyl, propyl, isopropyl, butyl, isobutyl, sec-butyl, tert-butyl, pentyl, neopentyl and hexyl. The term “C₁-C₆ alkenyl” refers to straight chain or branched chain hydrocarbon groups having from one to six carbon atoms and having in addition one or more double bonds, each of either E or Z stereochemistry where applicable, where this term would include for example, an alpha, beta-unsaturated methylene, vinyl, 1-propenyl, 1- and 2-butenyl and 2-methyl-2-propenyl, and where in a preferred embodiment the C₁-C₆ alkenyl group is a C₂-C₆ alkenyl group. The term “C₃-C₆ cycloalkyl” refers to an alicyclic group having from 3 to 6 carbon atoms, where illustrative cycloalkyl groups are cyclopropyl, cyclobutyl, cyclopentyl and cyclohexyl. The term “C₄-C₆ cycloalkenyl” refers to an alicyclic group having from 4 to 6 carbon atoms and having in addition one or more double bonds, where illustrative cycloalkenyl groups are cyclopentenyl, cyclohexenyl, cycloheptenyl and cyclooctenyl. The term “halogen” refers to fluorine, chlorine, bromine or iodine. The term “amino acid side chain” refers to a characteristic side chain attached to the —CH(NH₂)(COOH) moiety in the following R or S amino acids: glycine, alanine, valine, leucine, isoleucine, phenylalanine, tyrosine, tryptophan, serine, threonine, cystein, methionine, asparagine, glutamine, lysine, histidine, arginine, glutamic acid and aspartic acid.

Representative examples of hydroxamates, and methods for synthesizing hydroxamates are described in detail in U.S. Pat. Nos. 4,599,361, 4,720,486, 4,743,587, 4,996,358, 5,183,900, 5,189,178, 5,239,078, 5,240,958, 5,256,657, 5,300,674, 5,304,604, 5,310,763, 5,412,145, 5,442,110, 5,473,100, 5,514,677, 5,530,161, 5,643,964, 5,652,262, 5,691,382, 5,696,082, 5,700,838, 5,747,514 5,594,006, 5,763,621, 5,821,262, 5,840,939, 5,849,951, 5,859,253, 5,861,436, 5,866,717, 5,872,152, 5,902,791, 5,917,090, 5,919,940, 5,932,695, 5,962,521, 5,962,529, 6,017,889, 6,022,898, 6,028,110, 6,093,798, 6,103,739, 6,124,329, 6,124,332, 6,124,333 6,127,427, 6,218,389, 6,228,988, and 6,258,851. Representative foreign and international applications and publications include EP-A-0231081, EP-A-0236872, EP-A-0274453, EP-A-0489577, EP-A-0489579, EP-A-0497192, EP-A-0574758, and EP-A-0575844, as well as WO 90/05716, WO 90/05719, WO 91/02716, WO 92/09563, WO 92/17460, WO 92/13831, WO 92/22523, WO 93/09090, WO 93/09097, WO 93/20047, WO 93/24449, WO 93/24475, WO 94/02446, WO 94/02447, WO 94/21612, WO 94/21625, WO 94/24140, WO 94/25434, WO 94/25435, and WO 99/06361. Many hydroxamates are also readily available from a variety of commercial sources.

4. Polypeptide Inhibitors

Within other aspects of the invention polypeptide (including polypeptide derivative) inhibitors of matrix metalloproteinases can be utilized to extend the duration and utility of collagen. Representative examples of polypeptide inhibitors include those disclosed in U.S. Pat. Nos. 5,300,501, 5,530,128, 5,569,665, 5,714,491, and 5,889,058.-

5. Mercapto-Based Compounds

Mercapto-based compounds can also be utilized as MMPIs. Representative examples include mercaptoketon and mercaptoalcohol compounds such as those described in U.S. Pat. Nos. 5,831,004, 5,840,698, and 5,929,278; mercaptosulfides such as those described in U.S. Pat. No. 5,455,262.

6. Bisphosphonates

Bisphosphonates are compounds which are related to inorganic pyrophosphonic acid (see generally H. Fleisch, Endocr Rev., 19(1):80-100 (1998); see also, H. Fleisch, Bisphosphonates in Bone Disease: From the Laboratory to the Patient (1997, 3rd ed.). The Parthenon Publishing Group, New York and London). Generally, bisphosphonates have the structure: P—C—P. Particularly preferred bisphosphonates have the structure

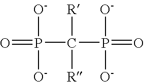

wherein the substituents R′ and R″ independently stand for a hydrogen or a halogen atom, a hydroxy, optionally substituted amino or optionally substituted thio group or an optionally substituted hydrocarbon residue. In one aspect, one of R′ and R″ is hydroxy, hydrogen or chlorine.

Representative examples of bisphosphonates include, for example, alendronate ((4-amino-1-hydroxybutylidene) bisphosphonic acid); clodronate (dichloromethane bisphosphonic acid); etidronate ((1-hydroxyethylidene) bisphosphonic acid); pamidronate ((3-amino-1-hydroxypropylidene) bisphosphonic acid); risedronate ([-hydroxy-2-(3-pyridinyl)ethylidene]bisphosphonic acid); tiludronate (([(4-chloro-phenyl)thio]-methylene]bisphosphonic acid); zolendronate; [1-hydroxy-3-(methyl-pentyl-amino)-propylidene]bis-phosphonate (BM21.0955); [(cycloheptylamino) methylene]-bisphos-phonate (YM175); 1-hydroxy-3-(1-pyrrolidinyl)-propylidene]bisphosphonate (EB-1053); [1-hydroxy-2-(1H-imidozol-1-yl)ethylidene]bisphosphonate (CGP 42'446) and (1-hydroxy-2-imidazo-[1,2-a]pyridin-3-yl-ethylidene) bisphosphonate (YM 529).

Representative examples of bisphosphonates are described in U.S. Patent Nos., 5,652,227 and 5,998,390.

7. Combinations of MMPIs

Within certain embodiments of the invention, more than one MMPI may be utilized (i.e., two or more MMPIs can be used in combination). Synergistic MMPIs include, for example tetracyclines and bisphosphonates (see, e.g., U.S. Pat. Nos. 5,998,390 and 6,114,316). Other combinations of MMPIs can likewise be utilized, including for example, MMPIs which inhibit MMPs at different stages (e.g., hydroxamates and tetracyclines).

III. Formulations

As noted above, collagen is a fibrous protein which can be obtained from natural sources or produced recombinantly. Representative examples of U.S. patents which described collagen-based compositions and methods of preparing such compositions include U.S. Pat. Nos. 6,166,130, 6,051,648, 5,874,500, 5,705,488, 5,550,187, 5,527,856, 5,523,291, 4,582,640, 4,424,208, and 3,949,073.

The MMPI compositions of the present invention can be prepared in a variety of ways. For example, the MMPI can be dissolved directly into the collagen solution. If the MMPI is stable in the collagen solution, the composition containing the collagen and the MMPI can be prepared in a single application apparatus. If the MMPI is not stable in the collagen solution for a significant length of time, the composition can be made as a two-component system in which the components are mixed immediately prior to use.

MMPI compositions of the present invention can also be generated by placing the MMPI factor in a carrier. Representative examples of carriers can include both polymeric and non-polymeric carriers (e.g., liposomes or vitamin-based carriers, and may be either biodegradable or non-biodegradable. Representative examples of biodegradable compositions include albumin, gelatin, starch, cellulose, dextrans, polysaccharides, fibrinogen, Poly(esters) [e.g., poly (D,L lactide), poly (D,L-lactide-co-glycolide), poly (glycolide), poly(e-caprolactone), copolymers and blends thereof] poly (hydroxybutyrate), poly (alkylcarbonate), poly(anhydrides) and poly (orthoesters) (see generally, Illum, L., Davids, S. S. (eds.) “Polymers in controlled Drug Delivery” Wright, Bristol, 1987; Arshady, J., Controlled Release 17:1-22 (1991); Pitt, Int. J. Pharm 59:173-196 (1990); Holland et al., J Controlled Release 4:155-0180 (1986)). Representative examples of nondegradable polymers include copolymers of ethylene oxide and propylene oxide [Pluronic polymers—BASF], EVA copolymers, silicone rubber, poly(methacrylate) based and poly(acrylate based polymers. Particularly preferred polymeric carriers include poly (D,L-lactic acid) oligomers and polymers, poly (L-lactic acid) oligomers and polymers, poly (glycolic acid), copolymers of lactic acid and glycolic acid, poly (caprolactone), poly (valerolactone), polyanhydrides, copolymers of caprolactone and/or lactic acid, and/or glycolic acid with polyethylene glycol or methoxypolyethylene glycol and blends thereof.

Polymeric carriers may be fashioned in a variety of forms, including for example, rod-shaped devices, pellets, slabs, or capsules (see, e.g., Goodell et al., Am. J. Hosp. Pharm. 43:1454-1461 (1986); Langer et al., “Controlled release of macromolecules from polymers”; in Biomedical polymers, Polymeric materials and pharmaceuticals for biomedical use, Goldberg, E. P., Nakagim, A. (eds.) Academic Press, pp. 113-137, 1980; Rhine et al., J. Pharm. Sci. 69:265-270 (1980); Brown et al., J. Pharm. Sci. 72:1181-1185 (1983); and Bawa et al., J Controlled Release 1:259-267 (1985)). MMPI factors may be linked by occlusion in the matrices of the polymer, bound by covalent linkages, or encapsulated in microcapsules. Within certain preferred embodiments of the invention, MMPI compositions are provided in non-capsular formulations such as microspheres (ranging from nanometers to micrometers in size), pastes, threads of various size, films and sprays.

Preferably, MMPI compositions of the present invention (which, within certain embodiments comprise one or more MMPI factors, and a polymeric carrier) are fashioned in a manner appropriate to the intended use. Within certain aspects of the present invention, the MMPI composition should be biocompatible, and release one or more MMPI factors over a period of several days to months. For example, “quick release” or “burst” MMPI compositions are provided that release greater than 10%, 20%, or 25% of an MMPI factor (e.g., tetracycline) over a period of 7 to 10 days. Such “quick release” compositions should, within certain embodiments, be capable of releasing chemotherapeutic levels (where applicable) of a desired MMPI factor. Within other embodiments, “low release” MMPI compositions are provided that release less than 5% (w/v) of an MMPI factor over a period of 7 to 10 days. Further, MMPI compositions of the present invention should preferably be stable for several months and capable of being produced and maintained under sterile conditions.

Within certain aspects of the present invention, MMPI compositions may be fashioned in any size ranging from about 0.050 nm to about 500 μm, depending upon the particular use. For example, when used for the purpose of cosmetic tissue augmentation (as discussed below), it is generally preferable to fashion the MMPI composition in microspheres of between about 0.1 to about 100 μm, preferably between about 0.5 and about 50 μm, and most preferably, between about 1 and about 25 μm. Alternatively such compositions may also be applied as a solution in which the MMPI is solubilized in a micelle. The composition of the micelles can be polymeric in nature. The most preferable polymeric composition for use as polymeric micelles would be a copolymer of MePEG and poly(D,L-lactide). Alternatively such compositions may also be applied as a solution in which the MMPI is encapsulated in a liposome (see above). Alternatively such compositions may also be applied as a solution in which the MMPI is encapsulated in the oil phase of an emulsion or microemulsion.

MMPI compositions of the present invention may also be prepared in a variety of “paste” or gel forms. For example, within one embodiment of the invention, MMPI compositions are provided which are liquid at one temperature (e.g., temperature greater than 37° C., such as 40° C., 45° C., 50° C., 55° C. or 60° C.), and solid or semi-solid at another temperature (e.g., ambient body temperature, or any temperature lower than 37° C.). Such “thermopastes” may be readily made given the disclosure provided herein.

Representative examples of the incorporation of MMPI factors such as those described above into a polymeric carriers is described in more detail below in the Examples.

Within further aspects of the present invention, polymeric carriers are provided which are adapted to contain and release a hydrophobic compound, the carrier containing the hydrophobic compound in combination with a carbohydrate, protein or polypeptide. Within certain embodiments, the polymeric carrier contains or comprises regions, pockets, or granules of one or more hydrophobic compounds. For example, within one embodiment of the invention, hydrophobic compounds may be incorporated within a matrix which contains the hydrophobic compound, followed by incorporation of the matrix within the polymeric carrier. A variety of matrices can be utilized in this regard, including for example, carbohydrates and polysaccharides such as starch, cellulose, dextran, methylcellulose, and hyaluronic acid, proteins or polypeptides such as albumin, collagen and gelatin. Within alternative embodiments, hydrophobic compounds may be contained within a hydrophobic core, and this core contained within a hydrophilic shell. For example, as described below in the Examples, paclitaxel may be incorporated into a hydrophobic core (e.g., of the poly D,L lactic acid-PEG or MePEG aggregate) which has a hydrophilic shell.

1. Collagen-MMP prodrugs

Within certain aspects of the present invention, MMPI compositions may be fashioned in such a manner that the MMPI is covalently attached to the collagen used in the specific application. The MMPI can be attached directly to the collagen or through a linker molecule (e.g., poly(ethylene glycol)). Once the collagen-MMP prodrug system is introduced/applied to the desired site, the MMPI could inhibit the MMP while still attached to the collagen or it could inhibit the MMP after it has been cleaved (hydrolytic and/or enzymatic cleavage) from the collagen.

For the TIMPs, a heterobifunctional crosslinking agent (e.g., Sulfo-EMCS [Pierce]) can be used to covalently bond the TIMP to the collagen. More specifically, the TIMP can be reacted with Sulfo-EMCS such that the maleimide group reacts with the —SH group of the cysteine contained with in the TIMP sequence. The activated TIMP can then be reacted with a collagen solution. The collagen-TIMP conjugate can then be used for tissue augmentation applications.

2. Further Compositions

Within certain embodiments of the invention, the collagen/MMPI compositions provided herein may be further modified in order to enhance their utility. For example, within one embodiment a dye or other coloring agent may be added to enhance visualization of the collagen/MMPI composition. The dye or coloring agent may be either permanent, or transient (e.g., methylene blue). Within other embodiments, compounds or factors which aid clotting (e.g., thrombin) may be added to the compositions described herein.

IV. Clinical Application

1. Dermal Injections

A variety of injectable collagen products have been developed for soft tissue augmentation to correct facial scars, diminish facial lines and augment the lips. Specifically, such implants are indicated for the treatment of a variety of contour deficiencies including (but not restricted to) correction of acne scars, atrophy from disease or trauma, glabellar frown lines, nasolabial folds, or defects secondary to rhinoplasty, skin graft or other surgery and other soft tissue defects.

Several commercially available products are used for this purpose, including Zyderm I® (3.5% bovine collagen in saline with 0.3% lidocaine), Zyderm II® (6.5% bovine collagen), Zyplast® (McGhan Medical Corporation; 3.5% bovine dermal collagen crosslinked with glutaraldehyde dispersed in phosphate-buffered physiologic saline containing 0.3% lidocaine) and Fibrel (Serono Labs—a combination of gelatin, epsilon-amino-caproic acid and saline combined with the patient's plasma in a 1:1 ratio prior to injection). Other collagen based injectable products, including those derived from non-bovine or human sources can be used in this embodiment as well.

Unfortunately, repeated “touch up” procedures are often required as the implant is colonized by host connective tissue cells and inflammatory cells which produce metalloproteinases, such as collagenase, that breaks down the collagen implant over time. An injectable collagen (as described above) containing a metalloproteinase inhibitor (MMPI), either alone or in a sustained release preparation, would result in increased durability of the implant and reduce the number of subsequent repeat injections.

Although any of the previously described metalloproteinase inhibitors could be suitable for incorporation into a dermal collagen injection, the following are particularly preferred: TIMP-1, tetracycline, doxycycline, minocycline, Batimistat®, Marimistat®, Ro-1130830, CGS 27023A, BMS-275291, CMT-3, Solimastat, Ilomastat, CP-544439, Prinomastat, PNU-1427690, SU-5402, and Trocade.

Regardless of the formulation utilized administration of the MMPI-loaded collagen injection would proceed in the following manner. Prior to administration of the material, the patient should have completed two skin tests (conducted 2 weeks apart) to test for an allergic response. If these tests are negative, the MMPI-loaded collagen injection can be administered to the patient. A refrigerated pre-loaded syringe with a fine gauge needle (30 or 32 gauge) containing no more than 309 cc's of the implant material is used. The patient is placed in a sitting position with the table back slightly reclined. Topical lidocaine and/or prilocaine can be used for anesthesia. The needle is inserted at an angle to the skin and advanced into the superficial dermal tissue. A sufficient amount of implant material is extruded to repair the soft tissue contour defect. In the case of MMPI-loaded Zyderm®, overcorrection (injection of more material than is ultimately needed) is required as a significant proportion of the injected material dissipates in the hours following injection. MMPI-loaded Zyplast® would typically be used to correct deeper lines and is injected deeper into the dermis. Since this material is more rigid, overcorrection is not required.

As described above, touch-up subsequent injections may be required to maintain maximum correction. However, a metalloproteinase inhibitor-loaded collagen injection will last longer than its unloaded counterpart, will provide long-standing correction and reduce the need for repeat injections.

The total amount of material injected is dependent upon the site of the contour deficiency being corrected; however, the total amount of material injected should not exceed 30 cc for a collagen-based product. The following MMPI-loaded compositions will be described on a dose per cc basis:

a. Marimistat®-Loaded Collagen Dermal Injections.

The preferred composition is 0.001%-30% Marimistat® per cc (i.e., 1 μg-30 mg Marimistat by weight) of collagen/saline suspension. A particularly preferred dosage is 0.01-1.5% Marimistat® (i.e., 10 μg to 1.5 mg) per cc of collagen/saline suspension. Therefore, the total dosage delivered in a 30 cc treatment would not exceed 45 mg (or less than the established well tolerated single daily does of 50 mg). In one embodiment, 0.001-30% Marimistat is loaded into PLGA microspheres or other polymer-based microspheres which are in turn loaded into the collagen, in order to produce sustained release of the material over a period ranging from several days to several months. Any source of injectable collagen (e.g., bovine, human, or recombinant; crosslinked or noncrosslinked) would be suitable to be combined with the above to produce the desired end product.

b. Batimistat®-Loaded Collagen Injections.

The preferred composition is 0.001 to 30% Batimistat® (i.e., 1 μg to 30 mg Batimistat® by weight) per cc of injectable collagen/saline suspension. A particularly preferred dosage is 0.01 to 5% (10 μg to 5 mg by weight) per cc of collagen/saline suspension. Therefore, the total dosage delivered in a 30 cc treatment would not exceed 150 mg of Batimistat® (or less than the established well tolerated single dose of 300 mg/m²). In one embodiment, the highly insoluble Batimistat® is loaded into PLGA microspheres or other polymer-based microspheres which are in turn loaded into the collagen, in order to produce sustained release of the agent over a period ranging from several days to several months. Any source of injectable collagen (e.g., bovine, human, or recombinant; crosslinked or noncrosslinked) would be suitable to be combined with the above to produce the desired end product.

c. Doxycycline-Loaded Collagen Dermal Injections.

The preferred composition is 0.001-30% doxycycline (1 μg to 30 mg doxycycline by weight) per cc of injectable collagen/saline suspension. A particular preferred dosage is 0.01 to 3% doxcycline (10 μg to 3 mg doxycycline by weight) per cc of collagen/saline suspension. Therefore the total dosage administered in a 30 cc treatment would not exceed 90 mg (or less than the well tolerated daily dosage of 100 mg). In one embodiment 0.001% to 30% doxycycline is loaded into PLGA microspheres or other polymer-based microspheres which are in turn loaded into the collagen, in order to produce sustained release of the agent over a period ranging from several days to several months. Any source of injectable collagen (e.g., bovine, human, or recombinant; crosslinked or noncrosslinked) would be suitable to be combined with the above to produce the desired end product.

d. Tetracycline-Loaded Dermal Collagen Injections

The preferred composition is 0.001-30% tetracycline (1 μg to 30 mg tetracycline by weight) per cc of injectable collagen/saline suspension. A particular preferred dosage is 0.01 to 30% tetracycline (10 μg to 30 mg tetracycline by weight) per cc of collagen/saline suspension. Therefore the total dosage administered in a 30 cc treatment would not exceed 900 mg (or less than the well tolerated daily dosage of 1 g). In one embodiment 0.001% to 30% tetracycline is loaded into PLGA microspheres or other polymer-based microspheres which are in turn loaded into the collagen, in order to produce sustained release of the agent over a period ranging from several days to several months. Any source of injectable collagen (e.g., bovine, human, or recombinant; crosslinked or noncrosslinked) would be suitable to be combined with the above to produce the desired end product.

e. Minocycline-Loaded Dermal Collagen Injections

The preferred composition is 0.001-30% minocycline (1 μg to 30 mg tetracycline by weight) per cc of injectable collagen/saline suspension. A particular preferred dosage is 0.01 to 6% minocycline (10 ug to 6 mg minocycline by weight) per cc of collagen/saline suspension. Therefore the total dosage administered in a 30 cc treatment would not exceed 180 mg (or less than the well tolerated daily dosage of 200 mg). In one embodiment 0.001% to 30% minocycline is loaded into PLGA microspheres or other polymer-based microspheres which are in turn loaded into the collagen, in order to produce sustained release of the agent over a period ranging from several days to several months. Any source of injectable collagen (e.g., bovine, human, or recombinant; crosslinked or noncrosslinked) would be suitable to be combined with the above to produce the desired end product.

f. Trocade-Loaded Dermal Collagen Injections

The preferred composition is 0.001-30% trocade (1 μg to 30 mg trocade by weight) per cc of injectable collagen/saline suspension. A particular preferred dosage is 0.01 to 5% trocade (10 μg to 5 mg trocade by weight) per cc of collagen/saline suspension. Therefore the total dosage administered in a 30 cc treatment would not exceed 150 mg. In one embodiment 0.001% to 30% trocade is loaded into PLGA microspheres or other polymer-based microspheres which are in turn loaded into the collagen, in order to produce sustained release of the agent over a period ranging from several days to several months. Any source of injectable collagen (e.g., bovine, human, or recombinant; crosslinked or noncrosslinked) would be suitable to be combined with the above to produce the desired end product.

2. Urinary Incontinence

Injectionable collagen is often used in the treatment of urinary incontinence. The embodiment described below details compositions of metalloproteinase inhibitor-loaded collagen products and methods for their use in the treatment of this common medical condition.

Briefly, incontinence, or the involuntary loss of urine, is a common medical condition which affects 20% of women and 1-2% of men at some point in their lifetime. The most common form of incontinence is stress incontinence, or the inadvertent leakage of urine in response to activities that cause an increase in intraabdominal pressure (such as sneezing, coughing, or straining). This occurs when intravesical pressure (pressure in the bladder) exceeds the pressure in the urethra, forcing urine from the bladder and into the urethra in the absence of detrusor (bladder muscle) contraction. Several conditions are thought to result in stress incontinence, including:

(1) Descent of the bladder neck and internal urethral spincter out of the abdomen.

(2) Intrinsic urethral spincter failure due to trauma, surgery, childbirth or malignancy.

Corrective measures are aimed principally at supporting the proximal urethral and bladder neck within the abdominal cavity by surgical or non-surgical means. A second approach involves the use of urethral bulking agents (including collagen) designed to increase urethral pressure and reduce stress incontinence.

Although periurethral and transurethral collagen injections have been used with a great deal of success in the management of stress incontinence, the majority of cases require more than one treatment due to the limited durability of the collagen implant. Utilizing a MMPI-loaded collagen injection can sustain the activity of the implant and reduce the need for, and frequency of, subsequent periurethral and transurethral injections.

Several commercially available collagen-based products are available for the management of stress incontinence. Contigeng (purified bovine dermal glutaraldehyde crosslinked collagen dispersed in phosphate buffered physiologic saline at 35 mg/ml available through CR Bard®) is a widely used urethral bulking agent. Other collagen based injectable products, including those derived from non-bovine, human, or recombinant sources can also be utilized in this embodiment. With Contigen®, the crosslinked collagen begins to degrade in approximately 12 weeks and degrades completely within 10 to 19 months. Although the percentage of patients showing improvement in their incontinence after therapy initially ranges from 58-100%, collagen resorption results in the need to repeat the procedure within the above mentioned time intervals in the majority of patients. In the present invention, an MMPI is added to the collagen-based injectable in a sustained-release form to decrease the rate of degradation of the implant and prolong its activity in vivo beyond that seen with collagen alone (i.e., consistently greater than 1 year in the majority of patients and beyond 2 years in a significant percentage of others).

Transurethral Technique:

Regardless of the formulation utilized, administration of a MMPI-loaded collagen transurethral injection would proceed in the following manner. Prior to administration of the material, the patient should have completed two skin tests (conducted 2 weeks apart) to test for an allergic response. If these tests are negative, the MMPI-loaded collagen injection can be administered to the patient. A refrigerated, single use, pre-loaded syringe with a fine gauge needle (23 guage transurethral injection needle with a stabilizing cannula) containing 2.5 ml of the implant material is used. The patient is placed in the lithotomy position and 10 ml of 2% lidocaine is inserted into the urethra for anesthesia. In women, the bladder neck is visualized cystoscopically. Via the injection port of the cystoscope, the needle is inserted at the 4 o'clock position, at a sharp angle, 1-1.5 cm distal to the bladder neck into the plane just beneath the bladder mucosa. The needle is then advanced with the cystoscope parallel to the long axis of the urethra until it lies just below the mucosa of the bladder neck. The MMPI-loaded collagen is injected slowly into this site. The procedure is then repeated at the 8 o'clock position. Methylene blue, or other nontoxic coloring agents, can be added to the implant to assist with visualization of the injection.

Periurethral Injection

Periurethral injection of an MMPI-loaded collagen injection can also be used for the treatment of incontinence. As described above, prior to administration of the material, the patient should have completed two skin tests (conducted 2 weeks apart) to test for an allergic response. If these tests are negative, the MMPI-loaded collagen injection can be administered to the patient. A refrigerated, single use, pre-loaded syringe with a fine gauge needle (periurethral injection needle) containing 2.5 mL of the implant material is used. The patient is placed in the lithotomy position, 10 mL of 2% lidocaine is inserted into the urethra for anesthesia and the bladder neck is visualized cystoscopically (in men the urethra can also be visualized via suprapubic cystooscopic approach). The needle is inserted transvaginally or suprapubically into the area immediately adjacent and lateral to the urethra. When it reaches the appropriate position near the bladder neck (as seen cystoscopically and described above), the MMPI-loaded collagen is injected slowly into this site. Methylene blue, or other nontoxic coloring agents, can be added to the implant to assist with visualization of the injection.

Although potentially any MMPI-loaded collagen injection could be suitable for transurethral or periurethral treatment of incontinence, MMPI's such as TIMP-1, tetracycline, doxycycline, minocycline, Batimistat®, Marimistat®, Ro-1130830, CGS 27023A, BMS-275291, CMT-3, Solimastat, Ilomastat, CP-544439, Prinomastat, PNU-1427690, SU-5402, and Trocade are particularly preferred. The following compositions are ideally suited for use as urinary bulking agents:

a. Marimistat®-Loaded Collagen Periurethral/Transurethral Injections.

The preferred composition is 0.001%-30% Marimistat® per cc (i.e., 1 μg-30 mg Marimistat by weight) of collagen/saline suspension. A particularly preferred dosage is 0.01-15% Marimistat® (i.e., 10 μg to 15 mg) per mL of collagen/saline suspension. Therefore, the total dosage delivered in a 2.5 mL treatment would not exceed 45 mg (or less than the established well tolerated single daily does of 50 mg). In one embodiment, 0.001-30% Marimistat is loaded into PLGA microspheres or other polymer-based microspheres which are in turn loaded into the collagen, in order to produce sustained release of the material over a period ranging from several days to several months. Any source of injectable collagen (e.g., bovine, human, or recombinant; crosslinked or noncrosslinked) would be suitable to be combined with the above to produce the desired end product.

b. Batimistat®-Loaded Collagen Periurethral/Transurethral Injections.

The preferred composition is 0.001 to 30% Batimistat® (i.e., 1 ug to 30 mg Batimistat® by weight) per mL of injectable collagen/saline suspension. A particularly preferred dosage is 0.01 to 30% (10 μg to 30 mg by weight) per mL of collagen/saline suspension. Therefore, the total dosage delivered in a 2.5 cc treatment would not exceed 75 mg of Batimistat® (or less than the established well tolerated single dose of 300 mg/m²). In one embodiment, 0.001 to 30% Batimistat® is loaded into PLGA microspheres or other polymer-based microspheres which are in turn loaded into the collagen, in order to produce sustained release of the agent over a period ranging from several days to several months. Any source of injectable collagen (e.g., bovine, human, or recombinant; crosslinked or noncrosslinked) would be suitable to be combined with the above to produce the desired end product.

c. Doxycycline-Loaded Collagen Periurethral/Transurethral Injections.

The preferred composition is 0.001-30% doxycycline (lug to 30 mg doxycycline by weight) per mL of injectable collagen/saline suspension. A particular preferred dosage is 0.01 to 30% doxcycline (10 μg to 30 mg doxycycline by weight) per mL of collagen/saline suspension. Therefore the total dosage administered in a 2.5 mL treatment would not exceed 75 mg (or less than the well tolerated daily dosage of 100 mg). In one embodiment 0.001% to 30% doxycycline is loaded into PLGA microspheres or other polymer-based microspheres which are in turn loaded into the collagen, in order to produce sustained release of the agent over a period ranging from several days to several months. Any source of injectable collagen (e.g., bovine, human, or recombinant; crosslinked or noncrosslinked) would be suitable to be combined with the above to produce the desired end product.

d. Tetracycline-Loaded Collagen Periurethral/Transurethral Injections

The preferred composition is 0.001-30% tetracycline (1 ug to 30 mg tetracycline by weight) per mL of injectable collagen/saline suspension. A particular preferred dosage is 0.01 to 30% tetracycline (10 μg to 30 mg tetracycline by weight) per mL of collagen/saline suspension. Therefore the total dosage administered in a 2.5 mL treatment would not exceed 75 mg (or less than the well tolerated daily dosage of 1 g). In one embodiment 0.001% to 30% tetracycline is loaded into PLGA microspheres or other polymer-based microspheres which are in turn loaded into the collagen, in order to produce sustained release of the agent over a period ranging from several days to several months. Any source of injectable collagen (e.g., bovine, human, or recombinant; crosslinked or noncrosslinked) would be suitable to be combined with the above to produce the desired end product.

e. Minocycline-Loaded Periurethral/Transurethral Collagen Injections

The preferred composition is 0.001-30% minocycline (lug to 30 mg tetracycline by weight) per cc of injectable collagen/saline suspension. A particular preferred dosage is 0.01 to 6% minocycline (10 μg to 6 mg minocycline by weight) per cc of collagen/saline suspension. Therefore the total dosage administered in a 30 cc treatment would not exceed 180 mg or less than the well tolerated daily dosage of 200 mg). In one embodiment 0.001% to 30% minocycline is loaded into PLGA microspheres or other polymer-based microspheres which are in turn loaded into the collagen, in order to produce sustained release of the agent over a period ranging from several days to several months. Any source of injectable collagen (e.g., bovine, human, or recombinant; crosslinked or noncrosslinked) would be suitable to be combined with the above to produce the desired end product.

f. Trocade-Loaded Collagen Periurethral/Transurethral Injections

The preferred composition is 0.001-30% trocade (1 ug to 30 mg trocade by weight) per mL of injectable collagen/saline suspension. A particular preferred dosage is 0.01 to 5% trocade (10 μg to 5 mg trocade by weight) per ml of collagen/saline suspension. Therefore the total dosage administered in a 2.5 mL treatment would not exceed 75 mg. In one embodiment 0.001% to 30% trocade is loaded into PLGA microspheres or other polymer-based microspheres which are in turn loaded into the collagen, in order to produce sustained release of the agent over a period ranging from several days to several months. Any source of injectable collagen (e.g., bovine, human, or recombinant; crosslinked or noncrosslinked) would be suitable to be combined with the above to produce the desired end product.

3. Surgical Sealants

Collagen has been widely used as a surgical sealant; particularly as a vascular sealant to stop bleeding following femoral puncture for vascular access and hemostasis during surgery.

Cannulation of the femoral artery is the initial step in gaining access to the vascular system as part of numerous common medical procedures such as coronary angiography, cerebral angiography, coronary angioplasty, coronary stenting, cerebral vascular aneurysm repair, abdominal aneurysm repair with stent grafts and several other procedures. For many of these indications relatively large devices must be introduced into the femoral artery necessitating a “cut down” procedure on the artery. Once the intervention has been completed and the catheter sheath withdrawn, it is often difficult to tamponade bleeding from the femoral puncture site (particularly since many of the patients are on anticoagulant therapy). Collagen-based vascular sealants have been developed for application over the puncture site to “seal” the wound and initiate healing of the arteriotomy. This can allow patients to ambulate sooner and prevent serious complications such as hematoma formation, or in severe cases, hemorrhage and significant blood loss. Hemostatic collagen sealants are also used to seal the adventitial (exterior) or cut surface of blood vessels, organs, bones and tissues during surgery as an adjunct to hemostasis when control of bleeding by ligature is not effective or possible. These products are used in cardiovascular, general, hepatic and orthopaedic surgical procedures.

Several collagen-based sealants are commercially available including Vasoseal™ (produced by Datascope®) and CoStasis™ (produced by Cohesion Technologies®). Producing an MMPI-loaded collagen-based sealant will prolong the activity of the collagen implant and allow the full healing process to occur prior to resorption of the implant. This may be of particular use in the control of surgical bleeding where it may not be possible to get easy access to a vascular repair site postoperatively.

Vasoseal™ is an example of a collagen “plug” kit for femoral artery puncture repair. Briefly, before removing the vascular procedural sheath, an ateriotomy locator is inserted into the sheath using an introducer. With the artery compressed, the procedural sheath and introducer are removed once the arteriotomy locator is correctly moved into position. A tissue dilator is advanced over the locator and a sheath is advanced over the dilator such that the sheath is positioned over the exterior surface of the arteriotomy site; the locator and dilator are then removed. The collagen cartridge (contains an 80-100 mg plug of purified bovine collagen) is inserted into the sheath and the collagen plug is injected over the puncture wound in the artery (2 injections may be required). An MMPI-loaded femoral artery sealant would be deployed in exactly the same manner, but would remain in place longer to allow complete healing to occur, thus reducing the risk of rebleeding at a later date. Examples of MMPI-loaded collagen plug formulations are provided below.

CoStasis™ is a sprayable liquid that is an example of a collagen-based hemostatic surgical sealant that would benefit from the addition of an MMPI (see, e.g., U.S. Pat. Nos. 5,290,552, 5,614,587, 5,744,545, 5,786,421, 5,936,035, 6,096,309, and 6,280,727). To deploy the system, the patient's own plasma is collected and drawn up into a syringe which is attached to a joining device. The collagen suspension (20 mg/mL bovine collagen and at least 300 U/ml bovine thrombin in a 40 mM CaCl₂ buffer) syringe is attached to the other port in the joiner. The joiner device mixes the contents of the collagen/thrombin syringe with the contents of the patient's plasma syringe. The bovine thrombin converts the autologous fibrinogen to fibrin, which in the presence of collagen, forms a collagen/fibrin gel matrix which adheres to the bleeding site. The mixture is then sprayed via the syringe over the bleeding site. The MMPI is added as a component of the collagen/thrombin suspension as described below. An MMPI-loaded collagen sealant would be of greatest utility in surgical procedures where prolonged hemostasis may be required until tissue healing occurs.

a. Marimistat®-Loaded Collagen Surgical Sealants.

The preferred composition is 0.001%-10% Marimistat® per ml (i.e., 1 ug-30 mg Marimistat by weight) of collagen/thrombin suspension (20 mg/ml bovine collagen and at least 300 U/ml bovine thrombin in a 40 mM CaCl₂ buffer). A particularly preferred dosage is 0.01-10% Marimistat® (i.e., 10 μg to 10 mg) per ml of collagen/thrombin suspension. Therefore, the total dosage delivered in a 5.0 ml treatment would not exceed 50 mg (or equal to the established well tolerated single daily does of 50 mg). In one embodiment, 0.001-10% Marimistat is loaded into PLGA microspheres or other polymer-based microspheres which are in turn loaded into the collagen/thrombin suspension, in order to produce sustained release of the material over a period ranging from several days to several months. Any source of injectable collagen (e.g., bovine, human, or recombinant; crosslinked or noncrosslinked) would be suitable to be combined with the above to produce the desired end product.

b. Batimistat®-Loaded Collagen Surgical Sealants.

The preferred composition is 0.001 to 30% Batimistat® (i.e., 1 μg to 30 mg Batimistat® by weight) per ml of injectable collagen/thrombin suspension (20 mg/ml bovine collagen and at least 300 U/mL bovine thrombin in a 40 mM CaCl₂ buffer). A particularly preferred dosage is 0.01 to 30% (10 μg to 30 mg by weight) per mL of collagen/thrombin suspension. Therefore, the total dosage delivered in a 5 ml treatment would not exceed 150 mg of Batimistat® (or less than the established well tolerated single dose of 300 mg/M²). In one embodiment, 0.001 to 30% Batimistat® is loaded into PLGA microspheres or other polymer-based microspheres which are in turn loaded into the collagen/thrombin suspension, in order to produce sustained release of the agent over a period ranging from several days to several months. Any source of injectable collagen (e.g., bovine, human, or recombinant; crosslinked or noncrosslinked) would be suitable to be combined with the above to produce the desired end product.

c. Doxycycline-Loaded Collagen Surgical Sealants.

The preferred composition is 0.001-20% doxycycline (1 μg to 30 mg doxycycline by weight) per ml of injectable collagen/thrombin suspension (20 mg/ml bovine collagen and at least 300 U/ml bovine thrombin in a 40 mM CaCl₂ buffer). A particular preferred dosage is 0.01 to 20% doxcycline (10 μg to 20 mg doxycycline by weight) per ml of collagen/thrombin suspension. Therefore the total dosage administered in a 5 mL treatment would not exceed 100 mg (or equal to the well tolerated daily dosage of 100 mg). In one embodiment 0.001% to 20% doxycycline is loaded into PLGA microspheres or other polymer-based microspheres which are in turn loaded into the collagen/thrombin suspension, in order to produce sustained release of the agent over a period ranging from several days to several months. Any source of injectable collagen (e.g., bovine, human, or recombinant; crosslinked or noncrosslinked) would be suitable to be combined with the above to produce the desired end product.

d. Tetracycline-Loaded Collagen Surgical Sealants

The preferred composition is 0.001-30% tetracycline (1 μg to 30 mg tetracycline by weight) per ml of injectable collagen/thrombin suspension (20 mg/ml bovine collagen and at least 300 U/ml bovine thrombin in a 40 mM CaCl₂ buffer). A particular preferred dosage is 0.01 to 30% tetracycline (10 μg to 30 mg tetracycline by weight) per mL of collagen/thrombin suspension. Therefore the total dosage administered in a 5 ml treatment would not exceed 150 mg (or less than the well tolerated daily dosage of 1 g). In one embodiment 0.001% to 30% tetracycline is loaded into PLGA microspheres or other polymer-based microspheres which are in turn loaded into the collagen/thrombin suspension, in order to produce sustained release of the agent over a period ranging from several days to several months. Any source of injectable collagen (e.g., bovine, human, or recombinant; crosslinked or noncrosslinked) would be suitable to be combined with the above to produce the desired end product.

e. Minocycline-Loaded Collagen Surgical Sealants

The preferred composition is 0.001-30% minocycline (1 μg to 30 mg tetracycline by weight) per ml of injectable collagen/thrombin suspension (20 mg/ml bovine collagen and at least 300 U/ml bovine thrombin in a 40 mM CaCl₂ buffer). A particular preferred dosage is 0.01 to 20% minocycline (10 μg to 20 mg minocycline by weight) per mL of collagen/thrombin suspension. Therefore the total dosage administered in a 5 ml treatment would not exceed 100 mg (or less than the well tolerated daily dosage of 200 mg). In one embodiment 0.001% to 30% minocycline is loaded into PLGA microspheres or other polymer-based microspheres which are in turn loaded into the collagen/thrombin suspension, in order to produce sustained release of the agent over a period ranging from several days to several months. Any source of injectable collagen (e.g., bovine, human, or recombinant; crosslinked or noncrosslinked) would be suitable to be combined with the above to produce the desired end product.

f. Trocade-Loaded Collagen Surgical Sealants

The preferred composition is 0.001-30% trocade (1 μg to 30 mg trocade by weight) per ml of injectable collagen/thrombin suspension (20 mg/ml bovine collagen and at least 300 U/ml bovine thrombin in a 40 mM CaCl₂ buffer). A particular preferred dosage is 0.01 to 10% trocade (10 μg to 10 mg trocade by weight) per mL of collagen/thrombin suspension. Therefore the total dosage administered in a 5 mL treatment would not exceed 50 mg. In one embodiment 0.001% to 30% trocade is loaded into PLGA microspheres or other polymer-based microspheres which are in turn loaded into the collagen/thrombin suspension, in order to produce sustained release of the agent over a period ranging from several days to several months. Any source of injectable collagen (e.g., bovine, human, or recombinant; crosslinked or noncrosslinked) would be suitable to be combined with the above to produce the desired end product.

g. Marimistat®-Loaded Collagen Femoral Puncture Sealants.

The preferred composition is 0.001%-10% Marimistat® (i.e., 1 μg-30 mg Marimistat by weight) per dose of collagen (80-100 mg collagen plug). A particularly preferred dosage is 0.01-10% Marimistat® (i.e., 10 μg to 10 mg) per collagen plug. In one embodiment, 0.001-10% Marimistat is loaded into PLGA microspheres or other polymer-based microspheres which are in turn loaded into the collagen, in order to produce sustained release of the material over a period ranging from several days to several months. Any source of injectable collagen (e.g., bovine, human, or recombinant; crosslinked or noncrosslinked) would be suitable to be combined with the above to produce the desired end product.

h. Batimistat®-Loaded Collagen Femoral Puncture Sealants.

The preferred composition is 0.001 to 30% Batimistat® (i.e., 1 μg to 30 mg Batimistat® by weight) per dose of collagen (80-100 mg collagen plug). A particularly preferred dosage is 0.01 to 30% (10 μg to 30 mg by weight) per collagen plug. In one embodiment, 0.001 to 30% Batimistat® is loaded into PLGA microspheres or other polymer-based microspheres which are in turn loaded into the collagen, in order to produce sustained release of the agent over a period ranging from several days to several months. Any source of injectable collagen (e.g., bovine, human, or recombinant; crosslinked or noncrosslinked) would be suitable to be combined with the above to produce the desired end product.

i. Doxycycline-Loaded Collagen Femoral Puncture Sealants.

The preferred composition is 0.001-20% doxycycline (1 μg to 30 mg doxycycline by weight) per dose of collagen (80-100 mg collagen plug). A particular preferred dosage is 0.01 to 20% doxcycline (10 μg to 20 mg doxycycline by weight) per collagen plug. In one embodiment 0.001% to 20% doxycycline is loaded into PLGA microspheres or other polymer-based microspheres which are in turn loaded into the collagen, in order to produce sustained release of the agent over a period ranging from several days to several months. Any source of injectable collagen (e.g., bovine, human, or recombinant; crosslinked or noncrosslinked) would be suitable to be combined with the above to produce the desired end product.

j. Tetracycline-Loaded Collagen Femoral Puncture Sealants

The preferred composition is 0.001-30% tetracycline (1 μg to 30 mg tetracycline by weight) per dose of collagen (80-100 mg collagen plug). A particular preferred dosage is 0.01 to 30% tetracycline (10 μg to 30 mg tetracycline by weight) per collagen plug. In one embodiment 0.001% to 30% tetracycline is loaded into PLGA microspheres or other polymer-based microspheres which are in turn loaded into the collagen, in order to produce sustained release of the agent over a period ranging from several days to several months. Any source of injectable collagen (e.g., bovine, human, or recombinant; crosslinked or noncrosslinked) would be suitable to be combined with the above to produce the desired end product.

k. Minocycline-Loaded Collagen Femoral Puncture Sealants

The preferred composition is 0.001-30% minocycline (1 μg to 30 mg tetracycline by weight) per dose of collagen (80-100 mg collagen plug). A particular preferred dosage is 0.01 to 20% minocycline (10 μg to 20 mg minocycline by weight) per collagen plug. In one embodiment 0.001% to 30% minocycline is loaded into PLGA microspheres or other polymer-based microspheres which are in turn loaded into the collagen, in order to produce sustained release of the agent over a period ranging from several days to several months. Any source of injectable collagen (e.g., bovine, human, or recombinant; crosslinked or noncrosslinked) would be suitable to be combined with the above to produce the desired end product.

l. Trocade-Loaded Collagen Femoral Puncture Sealants

The preferred composition is 0.001-30% trocade (1 μg to 30 mg trocade by weight) per dose of collagen (80-100 mg collagen plug). A particular preferred dosage is 0.01 to 10% trocade (10 μg to 10 mg trocade by weight) per collagen plug. In one embodiment 0.001% to 30% trocade is loaded into PLGA microspheres or other polymer-based microspheres which are in turn loaded into the collagen, in order to produce sustained release of the agent over a period ranging from several days to several months. Any source of injectable collagen (e.g., bovine, human, or recombinant; crosslinked or noncrosslinked) would be suitable to be combined with the above to produce the desired end product.

It should be readily evident to one of skill in the art that any of the previously mentioned MMPI's, or derivatives and analogues thereof, can be utilized to create variation of the above compositions without deviating from the spirit and scope of the invention.

### EXAMPLES

### Example 1

### Preparation of Collagen

Collagen Source

Skin is removed from freshly sacrificed rabbits. The removed skin is shaved, defatted by sharp dissection and cut into two cm² squares. The skin squares are freeze-dried at ambient temperature for 24 hours and then ground, with the aid of solid CO₂, in a mill to produce a powder.

Solubilization

A suspension of the powdered skin in prepared by adding the powdered material to a 0.5 M acetic acid solution such that the skin concentration is 5 g dry wt skin/l. The suspension is cooled to 10° C. A freshly prepared pepsin solution (0.5 g in 10 ml 0.01 N HCl) is added to the skin suspension and the mixture was incubated for 5 days at 110° C. with occasional stirring.

Pepsin Removal

Following the enzymatic treatment, the remaining pepsin in the mixture was denatured by adding 5 ml Tris base and adjusting the pH to 7.0 with 3 N NaOH at 4° C. 30 g NaCl is stirred into the mixture to keep the collagen in solution. After 4 hours, the mixture is centrifuged at 30,000 g for 30 minutes to remove the precipitated pepsin.

Purification

The enzymatically treated collagen is precipitated from the supernatant liquid by adding an additional 140 g NaCl. The solution is stirred and allowed to stand for 4 hours at 4° C. The precipitated collagen is centrifuged out at 30,000 g for 30 minutes. The resulting collagen pellet is resuspended in 200 ml deionized water. 0.5 N acetic acid is added to bring the final volume to one liter. The collagen is precipitated from this solution by adding 50 g NaCl, allowing the solution to stand for 5 hours at 4° C. and centrifuging at 30,000 g for 30 minutes.

Sterilization

The collagen pellet is resuspended in 200 ml distilled water, transferred into sterilized dialysis tubing and dialysed for 72 hours against 50 volumes 1 N acetic acid. The collagen was then dialysed for 24 hours against 50 volumes 0.001 N acetic acid with the solution being changed 3 times during this period. The dialysed solution is then concentrated by placing the dialysis tube on sterile absorbant towels in a laminar-flow bacteriologic barrier until the concentration reached 12-15 mg collagen/ml solution. The concentrated solution is then dialysed against 50 volumes 0.001 N acetic acid for 24 hours. The collagen solution is then stored in sterile vials at 4° C.

Addition of Polymerization Promoter to Concentrate

Immediately prior to use a buffered salt solution (NaCl 2.5 mM/l, NaHPO₄ 0.1 mM/l, pH 7.4) is added at 4° C. to the collagen solution in a volume: volume ratio of 10:1 (collagen:buffer), and the buffered concentrate is transferred to a chilled (4° C.) syringe. For specific applications (e.g., cosmetic tissue augmentation), the buffered salt solution can also contain 0.3-1% (w/v) of a local anesthetic (e.g., Lidocaine).

### Example 2

### Preparation of TIMP-1-Loaded Microspheres Using a W/O/W Method

Specifically, 100 mg of 50/50 PLGA copolymer (IV=0.15) is added to 12 mL of dichloromethane. To this, add 800 μL of phosphate buffered saline (PBS) solution or TIMP-1 (concentration typically from 1 to 10 mg/mL) in PBS. This mixture is then homogenized (20 seconds at 6,000 rpm). Once formed this mixture is dispersed into 100 mL of a 1.0% aqueous solution of poly vinyl alcohol (PVA) and is immediately homogenized (40 seconds at 8,000 rpm) to form a water in oil in water double emulsion. Polydisperse microparticles (with the majority less than 10 microns in size) are formed under these conditions. The solvent is then slowly removed via evaporation and the microspheres collected by centrifugation. The particles are washed (5 times) with deionized water and then frozen in a dry ice/acetone bath and lyophilized overnight to yield a white freely flowing powder of microspheres.

Microspheres with a longer degradation profile are prepared using 85/15 PLGA (IV=0.68) using the method described above.

The method described above is also used to prepare microspheres containing TIMP-2, TIMP-3 and TIMP-4.

### Example 3

### Preparation of Tetracycline-Loaded Microspheres Using a W/O/W Method

Tetracycline-loaded microspheres are prepared in a similar manner to that described in the example above except that tetracycline hydrochloride is used.

### Example 4

### Preparation of Doxycycline-Loaded Microspheres Using a W/O/W Method

Doxycycline-loaded microspheres are prepared in a similar manner to that described in the example above except that doxycycline hydrochloride is used.

### Example 5

### Preparation of Minocycline-Loaded Microspheres Using a W/O/W Method

Minocycline-loaded microspheres are prepared in a similar manner to that described in the example above except that minocycline hydrochloride is used.

### Example 6

### Preparation of Batimistat-Loaded Microspheres Using an Oil-In-Water Method

PVA Solution Preparation

In a 1000 ml beaker, 1000 ml of distilled water and 100 g of PVA (Aldrich 13-23K, 98% hydrolyzed) are added. A two-inch stirrer bar is placed into the beaker. The suspension is heated up to 75-80° C. while stirring. Once the PVA is dissolved completely (forms a clear solution), the PVA solution (w/v) is cooled to room temperature and filtered through a syringe in-line filter.

PLGA Solution Preparation with Batimistat

100 mg Batimistat and 900 mg PLGA (50/50, IV=0.15) are weighed and transferred into the 20 ml scintillation vial. 10 mL of HPLC grade dichloromethane (DCM) is added to the vial to dissolve the PLGA and Batimistat. The sample was place on an orbital shaker (setting 4) until the polymer and the Batimistat were dissolved.

Preparation of the Microspheres with Diameter Less than 25 μm

100 ml of 10% PVA solution is transferred into a 400 ml beaker. The beaker is secured to the stand using double-sided adhesive tape. A 3-blade stirring rod blades is placed into the beaker and adjusted to a height of approx. 0.5 cm above the beaker bottom. The stirrer motor (Dyna-Mix from Fisher Scientific) is turned on to 2.5 at first. The 10 ml PLGA/Batimistat solution is poured into the PVA solution during agitation. The stirring speed is the gradually increased to a setting of 5. The stirring is continued for 2.5 to 3.0 hours. The obtained microspheres were filtered through a 2 metal sieves (53 μm (top) and 25 μm (bottom)) into a 100 ml beaker in order to remove any large sized material. The microspheres are washed with distilled water while filtering. The microspheres that are collected in the filtrate were centrifuged (1000 rpm, 10 min.) to sediment the microspheres. The supernatant is removed using a Pasteur pipette and the pellet is re-suspended with 100 ml distilled water. This process is repeated 2 additional times.

The washed microspheres are transferred into a glass container. The transfer is completed by rinsing the beaker with a small amount of distilled water (20-30 ml). The container is sealed with Parafilm and placed into a −20° C. freezer over night. The frozen microsphere solution is then freeze-dried using a freeze-drier for about 3 days. The dried microspheres are transferred into 20 ml scintillation vial and were stored at −20° C. The microspheres are then terminally sterilized by irradiation with at least 2.5 Mrad Cobalt-60 (Co-60) x-rays.

### Example 7

### Preparation of Marmistat-Loaded Microspheres Using An Oil-in-Water Method

Marmistat-loaded microspheres are prepared in a similar manner to that described in the example above, except that Marmistat is used instead of Batimistat.

### Example 8

### Preparation of Trocade-Loaded Microspheres Using an Oil-in-Water Method

Trocade-loaded microspheres are prepared in a similar manner to that described in the example above, except that Trocade is used instead of Batimistat.

### Example 9

### Manufacture Collagen Solution Containing Micellar Batimistat

Preparation of the Polymer

Polymer is synthesized using DL-lactide and methoxy poly(ethylene glycol) [MePEG 2000] in presence of 0.5% w/w stannous octoate through a bulk ring opening polymerization.

Briefly, reaction glassware is washed and rinsed with Sterile Water for Irrigation USP, dried at 37° C., followed by depyrogenation at 250° C. for at least 1 hour. MePEG 2000 and DL-lactide are weighed (240 g and 160 g, respectively) and transferred to a round bottom flask using a stainless steel funnel. A 2-inch Teflon® coated magnetic stir bar is added to the flask. A glass stopper is used to seal the flask, which is then immersed, up to the neck, in a pre-heated oil bath. The oil bath is maintained at 140° C. using a temperature controlled hotplate. After the MePEG and DL-lactide have melted and reached 140° C., 2 mL of 95% stannous octoate (catalyst) is added to the flask. The flask is vigorously shaken immediately after the addition to ensure rapid mixing and is then returned to the oil bath. The reaction is allowed to proceed for 6 hours with heat and stirring. The liquid polymer is then poured into a stainless steel tray, covered and left in the fume hood overnight (about 16 hours). The polymer solidifies in the tray. The top of the tray is sealed using Parafilm®. The sealed tray containing the polymer is placed in a freezer at −20° C.±5° C. for 0.5 hour. The polymer is then removed from the freezer and transferred to glass storage bottles and stored at 2-8° C.

Preparation of Micellar Batimistat (Batimistat/Polymer Matrix)

Reaction glassware is washed and rinsed with Sterile Water for Irrigation USP, dried at 37° C., followed by depyrogenation at 250° C. for at least 1 hour. First, a phosphate buffer, 0.08M, pH 7.6 is prepared. The buffer is dispensed at the volume of 1 mL per vial. The vials are heated for 2 hours at 90° C. to dry the buffer. The temperature is then raised to 160° C. and the vials are dried for an additional 3 hours.

The polymer is dissolved in THF at 10% w/v concentration with stirring and heat. The polymer solution is then centrifuged at 3000 rpm for 30 minutes. The supernatant is poured off and set aside. Additional THF is added to the precipitate and centrifuged a second time at 3000 rpm for 30 minutes. The second supernatant is pooled with the first supernatant. Batimistat is weighed and then added to the supernatant pool. The solution is brought to the final desired volume with THF to make a 9.9% polymer solution containing 1.1% Batimistat.

To manufacture development batches of final product vials, the micellar Batimistat is dispensed into the vials containing dried phosphate buffer at a volume of 1 mL per vial. The vials are placed in a vacuum oven at 50° C. The vacuum is set at <-80 kPa and the vials remain in the oven overnight (15 to 24 hours). The vials are stoppered with Teflon faced gray butyl stoppers and sealed with aluminum seals. The Batimistat/polymer matrix is sterilized using 2.5 Mrad y-ray irradiation. Each vial contains approximately 11 mg Batimistat, 99 mg polymer, and 11 mg phosphate salts. The vials are stored at 2° to 8° C. until constitution.

Preparation of the Micellar Batimistat/Collagen Gel

In a sterile biological safety cabinet, two milliliters sterile saline is added to a vial that contained approximately 11 mg Batimistat, 99 mg polymer, and 11 mg phosphate salts (as prepared above). The contents of the vial are dissolved in 2 mL sterile saline by placing the vial in a water bath at 37° C. for approx. 30 minutes with periodic vortexing. Using a sterile 1 mL syringe, a 1 mL aliquot of the micellar Batimistat solution is withdrawn from the vial and was injected into 29 mL collagen gel. The sample is mixed to produce a homogeneous solution of the micellar Batimistat in the collagen gel. The sample is then loaded into 1 mL syringes for use in the in vivo experiments.

### Example 10

### Preparation of a 2 Component Micellar Kit

Preparation of Freeze Dried Micellar Batimistat

A solid composition capable of forming micelles upon constitution with an aqueous collagen-containing medium is prepared as follows:

Briefly, 41.29 g of MePEG (MW=2,000 g/mol) is combined with 412.84 g of 60:40 MePEG:poly(DL-lactide) diblock copolymer (see the example provided above) in a stainless steel beaker, heated to 75° C. in a mineral oil bath and stirred by an overhead stirring blade. Once a clear liquid is obtained, the mixture is cooled to 55° C. To the mixture is added a 200 ml solution of 45.87 g Batimistat in tetrahydrofuran. The solvent is added at approximately 40 ml/min and the mixture stirred for 4 hours at 55° C. After mixing for this time, the liquid composition is transferred to a stainless steel pan and placed in a forced air oven at 50° C. for about 48 hours to remove residual solvent. The composition is then cooled to ambient temperature and is allowed to solidify to form Batimistat-polymer matrix.

A phosphate buffer is prepared by combining 237.8 g of dibasic sodium phosphate heptahydrate, 15.18 g of monobasic sodium phosphate monohydrate in 1600 ml of water. To the phosphate buffer, 327 g of the Batimistat-polymer matrix is added and stirred for 2 hours to dissolve the solids. After a clear solution is achieved, the volume is adjusted to 2000 ml with additional water. Vials are filled with 15 ml aliquots of this solution and freeze dried by cooling to −34° C., holding for 5 hours, heating to −16° C. while reducing pressure to less than 0.2 mm Hg, holding for 68 hours, heating to 30° C. while maintaining low pressure, followed by holding for a further 20 hours. The result is a freeze-dried matrix that could constituted to form a clear micellar solution.

Preparation of 2 Component Kit

40 mg of the freeze-dried micellar Batimistat material is weighed into a capped 1 mL syringe. The plunger is replaced and the syringe is sealed in a plastic pouch using a heat sealer. The sample is sterilized using 2.5 Mrad γ-ray irradiation. Just prior to application, the plastic pouch containing the sterilized freeze-dried material is opened and connected to a dual syringe connector (Supplier, cat #). A syringe containing 2 mL 3.5% bovine collagen (95% type 1 and 5% Type III) is attached to the remaining end of the dual syringe connector. The plunger of the syringe containing the collagen material is pushed in order to transfer the collagen material into the syringe containing the micellar material. The material is passed rapidly from one syringe to the other until a homogeneous solution is obtained. The material is then transferred into the syringe that originally contained the collagen. This syringe is disconnected from the connector and a 30-gauge needle is connected to the syringe. The material is now ready for application.

### Example 11

### Preparation of a 2 Component Microsphere Kit

40 mg of the freeze-dried microsphere Batimistat material is weighed into a capped 1 mL syringe. The plunger is replaced and the syringe is sealed in a plastic pouch using a heat sealer. The sample is sterilized using 2.5 Mrad γ-ray irradiation. Just prior to application, the plastic pouch containing the sterilized freeze-dried material is opened and connected to a dual syringe connector. A syringe containing 2 mL 3.5% bovine collagen (95% type 1 and 5% Type III) is attached to the remaining end of the dual syringe connector. The plunger of the syringe containing the collagen material is pushed in order to transfer the collagen material into the syringe containing the micellar material. The material is passed from one syringe to the other until a homogeneous solution is obtained. The material is then transferred into the syringe that originally contained the collagen. This syringe is disconnected from the connector and a 30-gauge needle is connected to the syringe. The material is now ready for application.

### Example 12

### Liposomal Preparations

MLV Liposomes

A total of 100 mg of egg phosphatidylcholine (Avanti Polar Lipids) and cholesterol (Sigma) [5:1 molar ratio] are added to 5 mL dichloromethane in a 50 mL round bottom flask. Once dissolved, 3 mg Batimistat is added to the solution. The solvent is removed under slight vacuum using the rotavap. The lipid-drug mixture is dried overnight under vacuum. 5 mL 0.9% NaCl solution is added to the dried lipid-drug mixture. The solution is gently rotated for 1 hour using a rotavap and a water bath setting of 37° C. When 5% maltose is added to the 0.9% NaCl constitution solution, the samples are frozen in acetone dry ice and are freeze-dried to produce a solid product.

Depending on the specific dose required, a certain amount of the freeze-dried microsphere Batimistat material (prepared as described above) is weighed into a capped 1 mL syringe. The plunger is replaced and the syringe is sealed in a plastic pouch using a heat sealer. The sample is sterilized using 2.5 Mrad γ-ray irradiation. Just prior to application, the plastic pouch containing the sterilized freeze-dried material is opened and connected to a dual syringe connector. A syringe containing 3.5% bovine collagen (95% type 1 and 5% Type III) is attached to the remaining end of the dual syringe connector. The plunger of the syringe containing the collagen material is pushed in order to transfer the collagen material into the syringe containing the micellar material. The material is passed from one syringe to the other until a homogeneous solution is obtained. The material is then transferred into the syringe that originally contained the collagen. This syringe is disconnected from the connector and a 30-gauge needle is connected to the syringe. The material is now ready for application.

SUV Liposomes

The liposomes prepared above are size reduced by placing the sample in an ultrasonic bath (45° C.) for 10 minutes. The solution changed from a opaque—milky solution to a transparent solution with a blue tinge. This solution is either used as is or is freeze-dried to produce a solid product. The solid product can be used to prepare a collagen solution in a similar manner to that described above.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

We claim:

1. A composition comprising collagen and at least one metalloprotease inhibitor (MMPI).

2. The composition according to claim 1 wherein said MMPI is a Tissue Inhibitor of Matrix Metalloproteinase (TIMP).

3. The composition according to claim 2 wherein said TIMP is TIMP-1 or TIMP-2.

4. The composition according to claim 2 wherein said TIMP is TIMP-3 or TIMP-4.

5. The composition according to claim 1 wherein said MMPI is tetracycline, or an analog or derivative thereof.

6. The composition according to claim 5 wherein said MMPI is tetracycline.

7. The composition according to claim 6 wherein said tetracycline is minocycline or doxycline.

8. The composition according to claim 1 wherein said MMPI is a hydroxamate.

9. The composition according to claim 8 wherein said hydroxamate is Batimistat, Marimistat, or, Trocade.

10. The composition according to claim 1 wherein said MMPI is RO-1130830, CGS-27023A or BMS-275291.

11. The composition according to claim 1 wherein said MMPI is a polypeptide inhibitor.

12. The composition according to claim 11 wherein said polypeptide inhibitor is an inhibitor of a metalloprotease maturase.

13. The composition according to claim 1 wherein said MMPI is a mercapto-based compound.

14. The composition according to claim 1 wherein said MMPI is a bisphosphonate with structure (I): 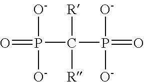
wherein R′ and R″ are independently a hydrogen, a halogen, a hydroxy, an optionally substituted amino group, an optionally substituted thio group, or an optionally substituted alkyl, alkanyl, alkenyl, alkynyl, alkyldiyl, alkyleno, heteroalkyl, heteroalkanyl, heteroalkenyl, heteroalkanyl, heteroalkyldiyl, heteroalkyleno, aryl, arylalkyl, heteroaryl, heteroarylalkyl.

15. The composition according to claim 14 wherein said MMPI is a bisphosphonate R′ and R″ is hydroxy, hydrogen, or chlorine.

16. The composition according to claim 1 comprising at least two MMPI.

17. The composition according to claim 16 wherein said at least two MMPI comprise a tetracycline, or an analog or derivative thereof and a bisphosphonate.

18. The composition according to claim 16 wherein said at least two MMPI comprise a tetracycline, or an analog or derivative thereof and a hydroxymate.

19. A composition comprising collagen, at least one metalloprotease inhibitor (MMPI), and at least one polymer.

20. The composition of claim 19 wherein said polymer is biodegradable.

21. The composition of claim 20 wherein said biodegradable polymer is selected from the group consisting of albumin, gelatin, starch, cellulose, dextrans, polysaccharides, fibrinogen, poly (esters), poly (D,L lactide), poly (D,L-lactide-co-glycolide), poly (glycolide), poly(e-caprolactone), poly (hydroxybutyrate), poly (alkylcarbonate), poly(anhydrides), and poly (orthoesters), and copolymers and blends thereof.

22. The composition of claim 19 wherein said polymer is a non-biodegradable polymer selected from the group consisting of an ethylene oxide and propylene oxide copolymer, an ethylene vinyl acetate copolymer, silicone rubber, a poly (methacrylate) based polymer, and a poly (acrylate) based polymer.

22. The composition of claim 20 wherein said polymer is in the form of a rod, a pellet, a slab, or a capsule.

23. The composition of claim 20 wherein said polymer is in the form of a microsphere, a paste, a thermopaste, a thread, a film, or a spray.

24. The composition of claim 19 wherein said MMPI is associated with the polymer by occlusion within the polymer matrix, by covalent linkage, or by encapsulation.

25. The composition of claim 1 wherein said MMPI is covalently linked to the collagen directly or via a linker.

26. The composition of claim 25 wherein said MMPI linked to the collagen is released by chemical cleavage or enzymatic cleavage of the covalent bond.

27. The composition of claim 19 further comprises a matrix, wherein said MMPI is incorporated within the matrix, said matrix being selected from the group consisting of carbohydrates, polysaccharides, starch, cellulose, dextran, methylcellulose, hyaluronic acid, polypeptides, albumin, collagen, and gelatin.

28. The composition according to claim 1 wherein said collagen is type I or type II.

29. The composition according to claim 19 wherein said collagen is type I or type II.

30. The compositions according to any one of claims 1 to 29, wherein said composition is sterile.

31. The composition according to any one of claims 1 to 29, further comprising thrombin or a dye.

32. The composition according to any one of claims 1 to 29, further comprising a pharmaceutically acceptable diluent, carrier, or excipient.

33. A method for the repair or augmentation of skin or tissue, comprising injecting into the skin or tissue a composition according to claim 30.

34. The method according to claim 33 wherein said injection is into the lips.

35. The method according to claim 33 wherein said injection is into the skin on the face.

36. A method for treating or preventing urinary incontinence, comprising administering to a patient a composition according to claim 30, such that said urinary continence is treated or prevented.

37. The method according to claim 36 wherein said composition is administered periurethrally.

38. The method according to claim 37 wherein said composition is administered transurethrally.

39. A method for sealing a surgical site, comprising, administering to a patent a composition according to claim 30.

40. The method according to claim 39 wherein said site is an area of vascular access.

41. A method for making collajolie, comprising admixing collagen and at least one MMPI.

42. The method according to claim 41, wherein said collagen is type I or type II collagen.

43. The method according to claim 41 wherein said MMPI is a Tissue Inhibitor of Matrix Metalloproteinase (TIMP).

44. The method according to claim 43 wherein said TIMP is TIMP-1 or TIMP-2.

44. The method according to claim 43 wherein said TIMP is TIMP-3 or TIMP-4.

45. The method according to claim 41 wherein said MMPI is tetracycline, or an analog or derivative thereof.

46. The method according to claim 45 wherein said MMPI is tetracycline.

47. The method according to claim 46 wherein said tetracycline is minocycline or doxycline.

48. The method according to claim 41 wherein said MMPI is a hydroxamate.

49. The method according to claim 48 wherein said hydroxamate is Batimistat, Marimistat, or, Trocade.

50. The method according to claim 41 wherein said MMPI is RO-1130830, CGS-27023A or BMS-275291.

51. The method according to claim 41 wherein said MMPI is a polypeptide inhibitor.

52. The method according to claim 51 wherein said polypeptide inhibitor is an inhibitor of a metalloprotease maturase.

53. The method according to claim 41 wherein said MMPI is a mercapto-based compound.

54. The method according to claim 41 wherein said MMPI is a bisphosphonate with structure (I): 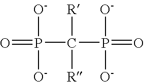
wherein R1 and R” are independently a hydrogen, a halogen, a hydroxy, an optionally substituted amino group, an optionally substituted thio group, or an optionally substituted alkyl, alkanyl, alkenyl, alkynyl, alkyldiyl, alkyleno, heteroalkyl, heteroalkanyl, heteroalkenyl, heteroalkanyl, heteroalkyldiyl, heteroalkyleno, aryl, arylalkyl, heteroaryl, heteroarylalkyl.

55. The method according to claim 54 wherein said MMPI is a bisphosphonate R′ and R″ is hydroxy, hydrogen, or chlorine.

56. The method according to claim 41 comprising at least two MMPI.

59. The method according to claim 41 wherein, prior to admixture with collagen, said MMPI is first admixed with at least one polymer.

60. The method of claim 59 wherein said polymer is biodegradable.

61. The method of claim 60 wherein said biodegradable polymer is selected from the group consisting of albumin, gelatin, starch, cellulose, dextrans, polysaccharides, fibrinogen, poly (esters), poly (D,L lactide), poly (D,L-lactide-co-glycolide), poly (glycolide), poly(e-caprolactone), poly (hydroxybutyrate), poly (alkylcarbonate), poly(anhydrides), and poly (orthoesters), and copolymers and blends thereof.

62. The method of claim 41, further comprising the step of sterilizing said mixture.
